(19) United States
(12) Patent Application Publication
(10) Pub. No.: US 2003/0219494 A1
(43) Pub. Date: Nov. 27, 2003

(54) Compositions and methods of using them

(76) Inventors: Maree Therese Smith, Queensland (AU); Lindsay Charles Brown, Sinnamon Park (AU); Mark Bradford Pullar Harvey, Queensland (AU); Craig McKenzie Williams, Queensland (AU)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/393,050

(22) Filed: Mar. 20, 2003

Related U.S. Application Data
(60) Provisional application No. 60/366,594, filed on Mar. 20, 2002.

(51) Int. Cl. A61K 33/00; A61K 31/704; A61K 31/7024; A61K 31/485; A61K 31/198; A61K 31/21

(52) U.S. Cl. 424/608; 514/23; 514/26; 514/282; 514/565; 514/509

(57) ABSTRACT

The present invention is directed to compositions and methods for inducing, promoting or otherwise facilitating pain relief. More particularly, the present invention relates to the use of a compound which either directly or indirectly prevents, attenuates or reverses the development of reduced opioid sensitivity, together with a compound which activates the opioid receptor that is the subject of the reduced opioid sensitivity, in methods and compositions for the prevention or alleviation of pain, especially in neuropathic conditions and even more especially in peripheral neuropathic conditions such as painful diabetic neuropathy (PDN).

### RELATED APPLICATION

This application claims the benefit of U.S. Provisional Application No. 60/366,594 filed Mar. 20, 2002, and which is hereby incorporated herein in its entirety by reference.

### FIELD OF THE INVENTION

THIS INVENTION relates generally to compositions and methods for inducing, promoting or otherwise facilitating pain relief. More particularly, the present invention relates to the use of a compound which either directly or indirectly prevents, attenuates or reverses the development of reduced opioid sensitivity, together with a compound which activates the opioid receptor that is the subject of the reduced opioid sensitivity, in methods and compositions for the prevention or alleviation of pain. Even more particularly, the present invention contemplates the use of two or more compounds in the provision of symptomatic relief of pain in pain-associated conditions, especially in neuropathic conditions and even more especially in peripheral neuropathic conditions such as painful diabetic neuropathy (PDN), in vertebrate animals and particularly in human subjects. The compounds may be provided alone or in combination with other compounds such as those that are useful in the control of neuropathic conditions, and especially of peripheral neuropathic conditions such as PDN. One embodiment of the present invention relates to the use of a nitric oxide donor and an opioid analgesic, especially a μ-opioid-receptor agonist or a κ₂-opioid receptor agonist, in the therapeutic management of vertebrate animals including humans, for the prevention or alleviation of pain. In another embodiment, the present invention encompasses a method for the production of analgesia in vertebrate animals including humans, comprising the simultaneous, sequential or separate administration of a nitric oxide donor and a μ-opioid receptor agonist, or a nitric oxide donor and a κ₂-opioid receptor agonist.

### BACKGROUND OF THE INVENTION

Painful diabetic neuropathy (PDN) is a common and debilitating complication of diabetes mellitus which causes numbness, weakness, tingling, heightened sensitivity, severe pain and loss of function in affected nerves, which can occur throughout the autonomic and somatic nervous systems. Between 40% and 60% of patients with diabetes develop mild to moderate PDN, and a further 5% to 10% develop a severe clinical condition that may necessitate surgical interventions such as amputation of digits or limbs. Clinical manifestations of PDN range from hyper-sensitivity to mild stimuli such as light pressure or touch (allodynia) to exaggerated responsiveness to a more intense stimulus (hyperalgesia) (Merskey, International Association for the Study of Pain. Elsevier 226 1986).

There are no preventative treatments for PDN (Sima et al. Diabetologia 42 773-88 1999), hence the therapeutic management of the condition is primarily palliative. This palliative management also represents a significant therapeutic obstacle, as the most efficient analgesic pharmaceuticals available, the μ-opioid receptor agonists, are ineffective in PDN. The mechanism of this opioid insensitivity is unclear, but investigations have shown that poor glycaemic control can reduce pain tolerance and pain threshold and thus reduce the effectiveness of analgesics such as morphine (Morley et al. Am J Med 77(1): 79-83 1984). In addition, there may be diabetes-associated alterations in morphine pharmacokinetics (Courteix et al. J Pharmacol Exp Ther 285(1): 63-70 1998) and/or changes in the affinity of opioid receptors for agonists.

There are several diabetic risk factors which predispose a patient to PDN, including poor metabolic control, dyslipidemia, body mass index and microalbuminuria, but these risk factors are not absolute: many patients with well-controlled diabetes will develop PDN and, conversely, many with poorly controlled diabetes will not develop the condition. Confounding observations such as these, in addition with disparity between animal and human models of diabetes have made the elucidation of the aetiology of PDN difficult. Presently, there are two broad theories regarding the development of the condition: the vascular dysfunction theory and the metabolic dysfunction theory.

The vascular dysfunction theory proposes that changes in the blood supply to the nerves (the neurovasculature or vasa nervorum) occur secondary to haemodynamic abnormalities (such as accelerated platelet aggregation and increased blood viscosity) (Fusman et al. Acta Diabetol 38(3):129-34 2001). In addition, pathological changes in the small blood vessels of the neurovasculature may occur (such as reduction of the production of nitric oxide from the endothelial cells of blood vessels and acceleration of the reactivity on vasoconstrictive substances) (McAuley et al. Clin Sci (Lond) 99(3): 175-9 2000). These haemodynamic and vascular changes, acting independently or synergistically, are capable of causing the perineurial ischaemia and subsequent endoneurial hypoxia observed in human patients and animal models of diabetes (Cameron et al. Diabetologia 44(11): 1973-88 2001). The end result of these abnormalities is nerve damage capable of causing the symptoms and signs of PDN.

On the other hand, in the metabolic dysfunction theory, the causes of nerve damage are mediated through the activation of the polyol metabolic pathway and through non-enzymatic protein glycation. These pathways induce mitochondrial and cytosolic NAD⁺/NADH redox imbalances and energy deficiencies in the nerves which can culminate in damage to neural and neurovascular tissues (Obrosova et al. FASEB J 16(1):123-5 2002). In addition, these metabolic changes are thought to activate protein kinase C (PKC) which is capable of heightening pain responses (Kamei et al. Expert Opin Investig Drugs 10(9): 1653-64 2001) and also of reducing opiate receptor sensitivity (Wang et al. Brain Res 723(1-2): 61-9 1996). Furthermore, heightened PKC activity is thought to reduce the binding affinity of μ-opioid receptors for ligands (Ohsawa et al. Brain Res 764 244-8 1998). The consequences of these metabolic abnormalities are nerve damage and reductions in opioid receptor sensitivity, as seen in PDN patients.

It is likely that neither theory is mutually exclusive and proponents of both theories converge in the belief that, downstream of vascular dysfunction or metabolic abnormalities, there is an imbalance in the production of vaso-active compounds in the vasa nervorum which leads to hypoxic ischaemia of diabetic nerves.

Of all the endogenous vasodilators, nitric oxide is the most potent and hence is a likely candidate for reduced synthesis and consequent diabetes-induced constrictions in vascular tone. As well as relaxing vascular smooth muscle, it also inhibits the processes of platelet aggregation, mitogenesis and proliferation of cultured vascular smooth muscle, and leucocyte adherence (Wroblewski et al. Prev Cardiol 3(4):172-177 2000). Nitric oxide is produced by the vascular endothelium by a group of enzymes called nitric oxide synthases. There are three isoforms of nitric oxide synthase (NOS) named according to their activity or the tissue type in which they were first described. These enzymes all convert the endogenous substrate, arginine, into citrulline, producing NO in the process.

In work leading up to the present invention, the inventors examined the utility of providing the nitric oxide donor L-arginine in an animal model of diabetic neuropathy to promote small vessel dilation in the vasa nervorum and discovered unexpectedly that the use of this amino acid rendered the animals opioid sensitive, thereby capacitating the relief of neuropathic pain with morphine. This discovery was indeed surprising in the light of prior evidence which had found that L-arginine attenuated the analgesic effects of opioids through alterations in uptake and distribution of morphine (Bhargava et al. Pharmacol Biochem Behav 61(1): 29-33 1998) and that inhibition of nitric oxide production was able to re-establish the analgesic physiological effects of morphine (Bian et al. Gen Pharmacol 30(5): 753-7 1998).

### SUMMARY OF THE INVENTION

The present invention is predicated in part on the determination that nitric oxide donors such as L-arginine can broadly prevent, attenuate and/or reverse the development of reduced analgesic sensitivity to an opioid receptor agonist, including the development of tolerance to an opioid receptor agonist resulting from the chronic administration of the agonist as well as the development of hyposensitivity to an opioid receptor agonist, which is associated with neuropathic conditions, and especially with peripheral neuropathic conditions such as PDN. Accordingly, the present invention in one aspect provides methods for producing analgesia in a subject having, or at risk of developing, reduced analgesic sensitivity to an opioid receptor agonist. In one embodiment, analgesia is produced by administering to the subject a nitric oxide donor in an amount that is effective for preventing, attenuating and/or reversing the reduced analgesic sensitivity. The nitric oxide donor is administered separately, simultaneously or sequentially with an opioid analgesic in an amount that is effective for producing the analgesia. Suitably, the opioid analgesic agonises the same opioid receptor as the opioid receptor agonist that is the subject of the reduced analgesic sensitivity. In one embodiment, the reduced analgesic sensitivity is associated with a neuropathic condition, including a peripheral neuropathic condition such as PDN or related condition. The nitric oxide donor and the opioid receptor agonist are suitably administered in the form of one or more compositions each comprising a pharmaceutically acceptable carrier and/or diluent. The composition(s) may be administered by injection, by topical application or by the oral route including sustained-release modes of administration, over a period of time and in amounts which are effective for the production of analgesia in the subject.

The nitric oxide donor is suitably selected from any substance that is converted into, or degraded or metabolised into, or provides a source of, in vivo nitric oxide. In one embodiment, the nitric oxide donor is L-arginine or an analogue or derivative thereof. In one embodiment, the opioid receptor agonist is a μ-opioid receptor agonist or a compound which is metabolised or otherwise converted in vivo to a μ-opioid receptor agonist. For example, the μ-opioid receptor agonist may be selected from morphine, methadone, fentanyl, sufentanil, alfentanil, hydromorphone, oxymorphone, their analogues, derivatives or prodrugs and a pharmaceutically compatible salt of any one of these. Suitably, the μ-opioid receptor agonist is morphine or an analogue or derivative or prodrug thereof, or a pharmaceutically compatible salt of these. In another embodiment, the opioid receptor agonist is a κ₂-opioid receptor agonist. Suitably, the κ₂-opioid receptor agonist is oxycodone or an analogue or derivative or prodrug thereof, or a pharmaceutically compatible salt of these.

In another aspect, the invention provides methods for producing analgesia in a subject having, or at risk of developing, reduced analgesic sensitivity to an opioid receptor agonist. In one embodiment, the analgesia is produced by administering to the subject L-arginine in an amount that is effective for preventing, attenuating and/or reversing the reduced analgesic sensitivity. The L-arginine is administered separately, simultaneously or sequentially with an opioid analgesic, which agonises the same opioid receptor as the opioid receptor agonist that is the subject of the reduced analgesic sensitivity, in an amount that is effective for producing the analgesia.

In another aspect, the invention provides analgesic compositions which generally comprise a nitric oxide donor and an opioid analgesic, each in an amount effective to produce analgesia in a subject. Typically, the subject exhibits or is at risk of developing reduced analgesic sensitivity to an opioid receptor agonist. In one embodiment of this type, the opioid analgesic agonises the same opioid receptor as the opioid receptor agonist that is the subject of the reduced analgesic sensitivity. In one embodiment, the nitric oxide donor is in association with the opioid analgesic, including the provision of the nitric oxide donor and opioid analgesic as separate compounds or in conjugate form. The nitric oxide donor and opioid receptor agonist are suitably in the form of pharmaceutically compatible salts and are present in effective amounts as broadly described above. In one embodiment, the compositions generally comprise L-arginine and an opioid analgesic, which agonises the same opioid receptor as an opioid receptor agonist that is the subject of reduced analgesic sensitivity. Suitably, the reduced analgesic sensitivity is associated with a neuropathic condition, including a peripheral neuropathic condition such as PDN or related condition.

In yet another aspect, the present invention contemplates the use of a nitric oxide donor and an opioid analgesic in the manufacture of a medicament for the production of analgesia in subjects. Suitably, the subjects have, or are at risk of developing, a neuropathic condition, including a peripheral neuropathic condition such as PDN or related condition. In one embodiment, the present invention encompasses the use of L-arginine and an opioid analgesic in the manufacture of a medicament for the production of analgesia in subjects.

### BRIEF DESCRIPTION OF THE DRAWINGS

FIG. 1 is a graphical representation showing the development and maintenance of mechanical allodynia (the defining symptom of PDN) for the 6-month study period in rats with STZ-induced diabetes. The time course of baseline paw withdrawal thresholds is shown for the left hindpaw for weight-matched control rats (n=6) and STZ-diabetic rats at 8 days (n=10), 3 (n=10), 9 (n=46), 12 (n=53), and 24 (n=36) wks post-STZ injection. Compared with the mean (±SEM) paw withdrawal threshold in non-diabetic control rats (11.9±0.2 g), the corresponding values determined in STZ-diabetic rats were significantly (p<0.05) lower, dropping to 8.0 (±0.3) g at 8 days and 5.2 (±0.3) g at 3 wks post-STZ. Thereafter, the baseline paw withdrawal thresholds remained relatively constant until 12 wks post-STZ (p>0.05). Between 12 and 24 wks post-STZ, there was a further small but significant decrease in the paw withdrawal threshold from 4.7 (±0.1) g to 3.3 (±0.1) g.

FIG. 2 is a graphical representation showing that the antinociceptive potency of morphine was completely abolished at 12 wks post-STZ administration. The mean (±SEM) dose-response curves are shown for s.c. morphine in diabetic rats at 3, 9, 12, and 24 wks post-STZ injection.

FIG. 3 is a graphical representation showing that the efficacy of oxycodone was maintained for the full 24 wk study period, albeit with a 4-fold decrease in antinociceptive potency at 12 wks which remained unchanged at 24 wks relative to control non-diabetic rats. The mean (±SEM) dose-response curves are shown for s.c. oxycodone in diabetic rats at 3, 9, 12, and 24 wks post-STZ injection.

FIG. 4 is a graphical representation showing that 3 wks of dietary L-arginine supplementation prevented the abolition of morphine's antinociceptive efficacy that occurred between 9 and 12 wks post-STZ administration. The mean (±SEM) antinociceptive dose-response curves are shown for s.c. morphine administered at 9, 12, and 24 wks post-STZ to adult male diabetic DA rats fed a standard rat chow diet or given the dietary L-arginine supplement from 9 wks to 24 wks post-STZ administration. Comparison is made with the dose response curve determined in non-diabetic control rats fed the dietary L-arginine supplement for 1 wk.

FIG. 5 is a graphical representation showing that 3 wks of dietary L-arginine supplementation prevented the 2-fold decrease in oxycodone potency that occurred between 9 and 12 wks post-STZ administration. The mean (±SEM) antinociceptive dose-response curves are shown for s.c. oxycodone administered at 9, 12, and 24 wks post-STZ to adult male diabetic DA rats fed a standard rat chow diet or given the dietary L-arginine supplement from 9 wks to 24 wks post-STZ administration. Comparison is made with the dose-response curve determined in non-diabetic control rats fed the dietary L-arginine supplement for 1 wk.

FIG. 6 is a graphical representation showing that dietary L-arginine supplementation in STZ-diabetic rats increased the potency of morphine for the relief of mechanical allodynia to ≈90% of that found in control non-diabetic rats. Specifically, this figure shows the mean (±SEM) degree of antinociception versus time curves following s.c. administration of morphine (5.45 and 6.1 mg/kg, n=7, 6, 5, 5, and 6, per dose) at 9, 12, 16, 20, and 24 wks post-STZ treatment in diabetic adult male DA rats with and without dietary L-arginine supplementation, respectively.

FIG. 7 is a graphical representation showing that dietary L-arginine supplementation increased the potency of oxycodone for the relief of mechanical allodynia to ≈150% of that found in diabetic rats fed a standard rat chow diet at 9 wks post-STZ. Specifically, this figure shows the mean (±SEM) degree of antinociception versus time curves following s.c. administration of the 9 wk post-STZ oxycodone ED₅₀ (2.0 mg/kg, n=7, 7, 6, and 4 per dose) at 9, 12, 20, and 24 wks post-STZ treatment in diabetic adult male DA rats with and without dietary L-arginine supplementation, respectively.

### DETAILED DESCRIPTION OF THE INVENTION

1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

As used herein, the term “about” refers to a quantity, level, value, dimension, size, or amount that varies by as much as 30%, 20%, or 10% to a reference quantity, level, value, dimension, size, or amount.

The term “allodynia” as used herein refers to pain that results from a non-noxious stimulus i.e., a stimulus that does not normally provoke pain. Examples of allodynia include, but are not limited to, cold allodynia, tactile allodynia (pain due to light pressure or touch), and the like.

The term “analgesia” is used herein to describe states of reduced pain perception, including absence from pain sensations as well as states of reduced or absent sensitivity to noxious stimuli. Such states of reduced or absent pain perception are induced by the administration of a pain-controlling agent or agents and occur without loss of consciousness, as is commonly understood in the art. The term analgesia encompasses the term “antinociception”, which is used in the art as a quantitative measure of analgesia or reduced pain sensitivity in animal models.

The term “causalgia” as used herein refers to the burning pain, allodynia and hyperpathia after a traumatic nerve lesion, often combined with vasomotor and sudomotor dysfunction and later tropic changes.

By “complex regional pain syndromes” is meant the pain that includes, but is not limited to, reflex sympathetic dystrophy, causalgia, sympathetically maintained pain, and the like.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

By “effective amount”, in the context of treating or preventing a condition is meant the administration of that amount of active to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

By “nitric oxide donor”, “NO donor” and the like is meant any substance that is converted into, degraded or metabolised into, or provides a source of in vivo nitric oxide or NO.

By “hyperalgesia” is meant an increased response to a stimulus that is normally painful.

By “neuropathic pain” is meant any pain syndrome initiated or caused by a primary lesion or dysfunction in the peripheral or central nervous system. Examples of neuropathic pain include, but are not limited to, thermal or mechanical hyperalgesia, thermal or mechanical allodynia, diabetic pain, entrapment pain, and the like.

“Nociceptive pain” refers to the normal, acute pain sensation evoked by activation of nociceptors located in non-damaged skin, viscera and other organs in the absence of sensitization.

The term “opioid-receptor agonist” as used herein refers to any compound which upon administration is capable of binding to an opioid receptor and causing agonism, partial agonism or mixed agonism/antagonism of the receptor. Metabolites of administered compounds are also encompassed by the term opioid receptor agonists. Preferred opioid receptor agonists are those that produce analgesia.

The term “pain” as used herein is given its broadest sense and includes an unpleasant sensory and emotional experience associated with actual or potential tissue damage, or described in terms of such damage and includes the more or less localised sensation of discomfort, distress, or agony, resulting from the stimulation of specialised nerve endings. There are many types of pain, including, but not limited to, lightning pains, phantom pains, shooting pains, acute pain, inflammatory pain, neuropathic pain, complex regional pain, neuralgia, neuropathy, and the like (Dorland's Illustrated Medical Dictionary, 28^{th} Edition, W. B. Saunders Company, Philadelphia, Pa.). The goal of treatment of pain is to reduce the severity of pain perceived by a treatment subject.

By “pharmaceutically acceptable carrier” is meant a solid or liquid filler, diluent or encapsulating substance that may be safely used in topical, local or systemic administration.

The term “pharmaceutically compatible salt” as used herein refers to a salt which is toxicologically safe for human and animal administration. This salt may be selected from a group including hydrochlorides, hydrobromides, hydroiodides, sulphates, bisulphates, nitrates, citrates, tartrates, bitartrates, phosphates, malates, maleates, napsylates, fumarates, succinates, acetates, terephthalates, pamoates and pectinates.

The term “prodrug” is used in its broadest sense and encompasses those compounds that are converted in vivo to an opioid receptor agonist according to the invention. Such compounds would readily occur to those of skill in the art, and include, for example, compounds where a free hydroxy group is converted into an ester derivative. Prodrug forms of compounds may be utilised, for example, to improve bioavailability, mask unpleasant characteristics such as bitter taste, alter solubility for intravenous use, or to provide site-specific delivery of the compound.

The terms “reduced opioid analgesic sensitivity”, “reduced analgesic sensitivity to an opioid receptor agonist” and the like are used interchangeably herein to refer to an abrogated, impaired or otherwise reduced analgesia produced by the administration of an amount or concentration of an opioid receptor agonist, which would otherwise produce analgesia in an opioid-naïve individual, especially in an opioid-naïve individual who does not have a neuropathic pain condition, more especially in an opioid-naïve individual who does not have a peripheral neuropathic pain condition and even more especially in an opioid-naïve non-diabetic individual.

The terms “subject” or “individual” or “patient”, used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, primates, avians, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g. foxes, deer, dingoes). A preferred subject is a human in need of treatment or prophylaxis for a peripheral neuropathic condition, especially PDN. However, it will be understood that the aforementioned terms do not imply that symptoms are present.

2. Methods for the Production of Analgesia

The present invention provides methods for producing analgesia in a subject having, or at risk of developing, reduced analgesic sensitivity to an opioid receptor agonist. These methods generally comprise administering separately, simultaneously or sequentially to the subject a nitric oxide donor and an opioid analgesic, which agonises the same receptor as the opioid receptor agonist that is the subject of the reduced analgesic sensitivity. The nitric oxide donor is administered in an amount that is effective for preventing, attenuating and/or reversing the reduced analgesic sensitivity to the opioid receptor agonist whereas the opioid receptor agonist is administered in an amount that is effective for producing the analgesia, which effectiveness has been capacitated or otherwise rendered possible by the administration of the nitric oxide donor. The nitric oxide donor and the opioid receptor agonist are suitably in association with a pharmaceutically acceptable carrier and/or diluent, and may be administered separately or in combination with each other.

The reduced analgesic sensitivity may relate to the development of tolerance to an opioid receptor agonist, which results from the chronic administration of that agonist. In one embodiment, the reduced analgesic sensitivity is associated with a neuropathic condition and thus, the method of the present invention has particular utility in the prevention and/or alleviation of the painful symptoms associated with neuropathic conditions. There are many possible causes of neuropathic conditions and it will be understood that the present invention contemplates the treatment and/or prevention of pain associated with any neuropathic condition regardless of the cause. In one embodiment, the neuropathic conditions are a result of diseases of the nerves (primary neuropathy) and neuropathy that is caused by systemic disease (secondary neuropathy), such as but not limited to diabetic neuropathy, Herpes Zoster (shingles)-related neuropathy, uraemia-associated neuropathy, amyloidosis neuropathy, HIV sensory neuropathies, hereditary motor and sensory neuropathies (HMSN), hereditary sensory neuropathies (HSNs), hereditary sensory and autonomic neuropathies, hereditary neuropathies with ulcero-mutilation, nitrofurantoin neuropathy, tumaculous neuropathy, neuropathy caused by nutritional deficiency and neuropathy caused by kidney failure. Other causes include repetitive activities such as typing or working on an assembly line, medications known to cause peripheral neuropathy such as several AIDS drugs (DDC and DDI), antibiotics (metronidazole, an antibiotic used for Crohn's disease, isoniazid used for tuberculosis), gold compounds (used for rheumatoid arthritis), some chemotherapy drugs (such as vincristine and others) and many others. Chemical compounds are also known to cause peripheral neuropathy including alcohol, lead, arsenic, mercury and organophosphate pesticides. Some peripheral neuropathies are associated infectious processes (such as Guillian-Barre syndrome). In another embodiment, the neuropathic condition is a peripheral neuropathic condition such as PDN or related condition.

The neuropathic condition may be acute or chronic and, in this connection, it will be understood by persons of skill in the art that the time course of a neuropathy will vary, based on its underlying cause. With trauma, the onset of symptoms may be acute, or sudden, with the most severe symptoms being present at the onset or developing subsequently. Inflammatory and some metabolic neuropathies have a subacute course extending over days to weeks. A chronic course over weeks to months usually indicates a toxic or metabolic neuropathy. A chronic, slowly progressive neuropathy over many years occurs with most hereditary neuropathies or with a condition termed chronic inflammatory demyelinating polyradiculoneuropathy (CIDP). Neuropathic conditions with symptoms that relapse and remit include the Guillian-Barre syndrome.

Advantageously, the nitric oxide donor and the opioid receptor agonist are administered with compositions having other useful anti-neuropathic properties or compounds which otherwise facilitate amelioration of the symptoms and signs of the neuropathic condition of interest.

Not wishing to be bound by any one particular theory or mode of operation, it is proposed that nitric oxide donors induce a direct or indirect physiological effect on opioid receptors to render them capable of being activated by their cognate opioid-receptor agonists, thereby producing antinociception/analgesia. Thus, in another embodiment, the invention provides methods for producing analgesia in a subject having, or at risk of developing, a condition associated with opioid receptor hyposensitivity, wherein the methods generally comprise administering separately, simultaneously or sequentially to the subject a nitric oxide donor in an amount that is effective for rendering the opioid receptor capable of being activated by a cognate opioid receptor agonist, together with the cognate opioid receptor agonist in an amount that is effective for activating the receptor and producing analgesia in the subject.

The nitric oxide donor includes and encompasses any substance that is converted into, or degraded or metabolised into, or provides a source of, in vivo nitric oxide. This category includes compounds having differing structural features. For example, the nitric oxide donor includes, but is not limited to, L-arginine, sodium nitroprusside, nitroglycerine, glyceryl trinitrate, isosorbide mononitrate, isosorbide dinitrate, S-nitroso-N-acetyl-penicillamine, pseudojujubogenin glycosides such as dammarane-type triterpenoid saponins (e.g. bacopasaponins) as well as their derivatives or analogues. In one embodiment, the nitric oxide donor is L-arginine or an analogue or derivative thereof. Thus, in another aspect, the invention provides a method for producing analgesia in a subject having, or at risk of developing, reduced analgesic sensitivity to an opioid receptor agonist, comprising the separate, simultaneous or sequential administration to the subject of an effective amount of L-arginine or an analogue or derivative thereof, and an effective amount of an opioid analgesic, which agonises the same the opioid receptor agonist that is the subject of the reduced analgesic sensitivity.

In one embodiment, the opioid analgesic is a μ-opioid receptor agonist or a compound that is metabolised or otherwise converted in vivo to a μ-opioid receptor agonist. For example, the μ-opioid receptor agonist may be selected from morphine, methadone, fentanyl, sufentanil, alfentanil, hydromorphone, oxymorphone, their analogues, derivatives or prodrugs and pharmaceutically compatible salts of these. Suitably, the μ-opioid receptor agonist is morphine or an analogue or derivative or prodrug thereof or a pharmaceutically compatible salt of these. In another embodiment, the opioid analgesic is a κ₂-opioid receptor agonist. The κ₂-opioid receptor agonist may be metabolised or otherwise converted in vivo to a μ-opioid receptor agonist. Suitably, the κ₂-opioid receptor agonist is any compound which upon administration is capable of binding to a κ₂-opioid receptor and causing agonism, partial agonism or mixed agonism/antagonism of that receptor, and whose antinociceptive effects are attenuated or otherwise impaired by nor-BNI (nor-binaltorphimine; a putatively selective κ₁/κ₂-opioid receptor ligand) and which does not displace the binding of the κ₂-selective radioligand, [³H]U69,593, from rat brain membranes. Metabolites of administered compounds are also encompassed by the term opioid receptor agonists. Suitably, the κ₂-opioid receptor agonist is oxycodone or an analogue or derivative or prodrug thereof or a pharmaceutically compatible salt of these.

The nitric oxide donor and opioid analgesic may be provided either as separate compounds or in conjugate form. Conjugates, which are contemplated by the present invention, include at least one nitric oxide donor that is linked or coupled to, or otherwise associated with, at least one opioid analgesic. In one embodiment, the conjugate comprises an opioid receptor agonist that is coupled to nitrato group by a suitable linker. Exemplary conjugates of this type include, but are not limited to:

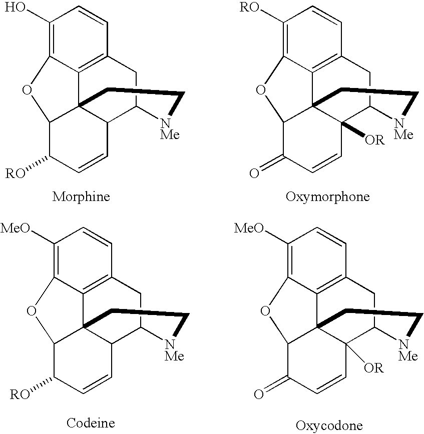

wherein R is H or a group represented by the formula:

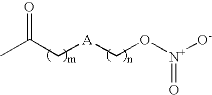

where A is absent or represents a group —O—, —S—, —NH—, —C₆H₄—, —OC₆H₄—, —SC₆H₄— or —NHC₆H₄—;

m is 0 or an integer from 1 to 10; and

n is an integer from 1 to 10 or when A is absent and m is 0, n is an integer from 3 to 10,

and their pharmaceutically compatible salts.

Suitably, R is a group represented by a formula selected from the group:

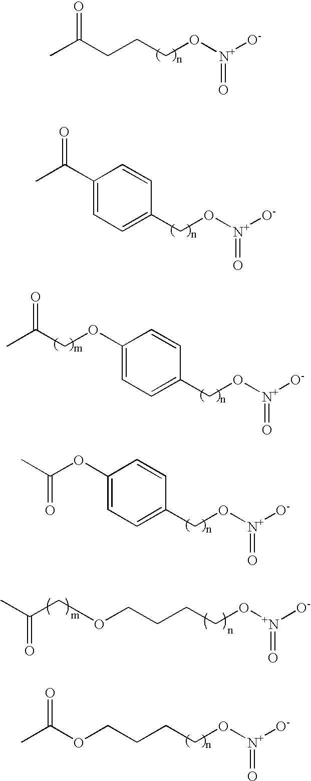

In embodiments of the present invention, the conjugate is a compound represented by a formula selected from the following group:

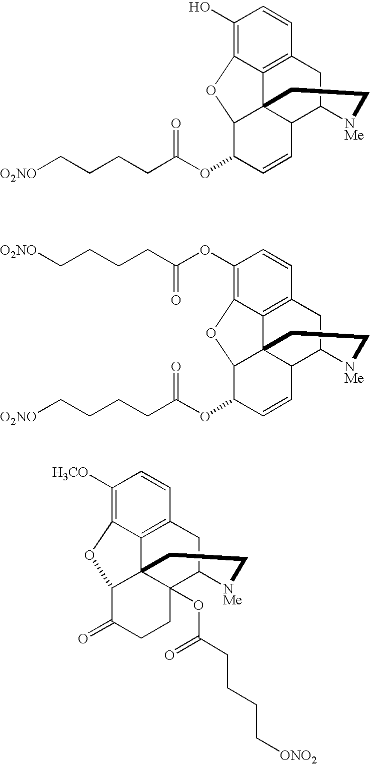

and their pharmaceutically compatible salts.

An effective amount of a nitric oxide donor is one that is effective for preventing, attenuating and/or reversing the reduced analgesic sensitivity, for restoring the analgesic sensitivity to a pre-existing level of sensitivity and includes the prevention, attenuation and/or reversal of the development of analgesic hyposensitivity to an opioid receptor agonist, which is associated with a neuropathic condition, including a peripheral neuropathic condition such as PDN or a related condition. An effective amount of an opioid receptor agonist is one which has been rendered effective by the nitric oxide donor for the treatment or prevention of pain in pain-associated conditions, including the prevention of incurring pain, holding pain in check, and/or treating existing pain. The pain may be associated with any pain associated condition, including cancer and neuropathic conditions, and especially peripheral neuropathic conditions such as PDN. Modes of administration, amounts of nitric oxide donor and opioid receptor agonist administered, and formulations, for use in the methods of the present invention, are discussed below.

Whether pain has been treated is determined by measuring one or more diagnostic parameters which is indicative of pain (e.g., subjective pain scores, tail-flick tests and tactile allodynia) compared to a suitable control. In the case of an animal experiment, a “suitable control” is an animal not treated with the nitric oxide donor and/or with the opioid receptor agonist, or treated with the pharmaceutical composition without nitric oxide donor and/or without the opioid receptor agonist. In the case of a human subject, a “suitable control” may be the individual before treatment, or may be a human (e.g., an age-matched or similar control) treated with a placebo. In accordance with the present invention, the treatment of pain includes and encompasses without limitation: (i) preventing pain experienced by a subject which may be predisposed to the condition but has not yet been diagnosed with the condition and, accordingly, the treatment constitutes prophylactic treatment for the pathologic condition; (ii) inhibiting pain initiation or a painful condition, i.e., arresting its development; (iii) relieving pain, i.e., causing regression of pain initiation or a painful condition; or (iv) relieving symptoms resulting from a disease or condition believed to cause pain, e.g., relieving the sensation of pain without addressing the underlying disease or condition.

3. Compositions

Another aspect of the present invention provides compositions for producing analgesia and especially for treating, preventing and/or alleviating the painful symptoms of a neuropathic condition. These analgesic compositions generally comprise a nitric oxide donor that is effective for preventing, attenuating or reversing the development of reduced analgesic sensitivity to an opioid receptor agonist, and an opioid analgesic. Suitably, the opioid analgesic agonises the same receptor as the opioid receptor agonist that is the subject of the reduced opioid sensitivity and is present in an amount that is effective for producing analgesia in the subject.

Any known nitric oxide donor and/or opioid receptor agonist compositions can be used in the methods of the present invention, provided that the nitric oxide donor and/or opioid analgesic are pharmaceutically active. A “pharmaceutically active” nitric oxide donor is in a form which results in preventing, attenuating or reversing the development of reduced analgesic sensitivity to an opioid receptor agonist, e.g. prevents, attenuates or reverses the development of hyposensitivity to an opioid receptor agonist that is associated with a neuropathic condition. A “pharmaceutically active” opioid analgesic is in a form which activates, or which has been rendered capable of activating, or is metabolised or converted in vivo to be capable of activating, the corresponding opioid receptor.

The effect of compositions of the present invention may be examined by using one or more of the published models of pain/nociception or of neuropathy, especially peripheral neuropathy, and more especially PDN, known in the art. This may be demonstrated, for example using a model which assesses the onset and development of hyperalgesia or tactile allodynia, the defining symptom of PDN, as for example described herein. The analgesic activity of the compounds of this invention can be evaluated by any method known in the art. Examples of such methods are the Tail-flick test (D'Amour et al. 1941, J. Pharmacol. Exp. and Ther. 72: 74-79); the Rat Tail Immersion Model, the Carrageenan-induced Paw Hyperalgesia Model, the Formalin Behavioral Response Model (Dubuisson et al., 1977, Pain 4: 161-174), the Von Frey Filament Test (Kim et al., 1992, Pain 50: 355-363), the Chronic Constriction Injury, the Radiant Heat Model, and the Cold Allodynia Model (Gogas et al., 1997, Analgesia 3: 111-118), the poor pressure test (Randall and Selitto, 1997, Arch Int Pharmacodyn 111: 409-414), and the paw pressure test (Hargreaves et al., 1998, Pain, 32: 77-88). An in vivo assay for measuring the effect of test compounds on the tactile allodynia response in neuropathic rats is described in Example 2. Compositions which test positive in such assays are particularly useful for the prevention, reduction, or reversal of opioid hyposensitivity in a variety of pain-associated conditions or pathologies including cancer, and are especially useful for the prevention, reduction, or reversal of opioid hyposensitivity secondary to neuropathic pain found, for example, in diabetic patients.

The active compounds of the present invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

Pharmaceutical compositions suitable for use in the present invention include compositions wherein the pharmaceutically active compounds are contained in an effective amount to achieve their intended purpose. The dose of active compounds administered to a patient should be sufficient to achieve a beneficial response in the patient over time such as a reduction in, or relief from, pain. The quantity of the pharmaceutically active compounds(s) to be administered may depend on the subject to be treated inclusive of the age, sex, weight and general health condition thereof. In this regard, precise amounts of the active compound(s) for administration will depend on the judgement of the practitioner. In determining the effective amount of the active compound(s) to be administered in the production of analgesia, the physician may evaluate severity of the pain symptoms associated with nociceptive or inflammatory pain conditions or numbness, weakness, pain, loss of reflexes and tactile allodynia associated with neuropathic conditions, especially peripheral neuropathic conditions such as PDN. In any event, those of skill in the art may readily determine suitable dosages of the nitric oxide donors and/or the opioid receptor agonists of the invention without undue experimentation.

In one embodiment, and dependent of the intended mode of administration, the nitric oxide donor-containing compositions will generally contain about 0.1% to 90%, about 0.5% to 50%, or about 1% to about 25%, by weight of nitric oxide donor, the remainder being suitable pharmaceutical carriers and/or diluents etc and optionally an opioid receptor agonist. Usually, a daily dose of nitric oxide donor may be from about 5 to 250 mg per day, from about 10 to 150 mg or from 20 to 120 mg for isosorbide dinitrate. The dosage of the nitric oxide donor can depend on a variety of factors, such as the individual nitric oxide donor, mode of administration, the species of the affected subject, age and/or individual condition. Normally, in the case of oral administration, an approximate daily dose of from about 10 mg to about 5000 mg, for the case of L-arginine or about 200 mg to 2000 mg per day, suitably 500 mg to 1000 mg per day is to be estimated for an adult patient of approximately 75 kg in weight.

In another embodiment, and dependent on the intended mode of administration, the opioid receptor agonist-containing compositions will generally contain about 0.1% to 90%, about 0.5% to 50%, or about 1% to about 25%, by weight of opioid receptor agonist, the remainder being suitable pharmaceutical carriers and/or diluents etc and optionally a nitric oxide donor. Usually, a daily oral dose of morphine in an opioid-naïve adult human may be from about 10 mg to 300 mg per day, from about 20 mg to 200 mg per day, or from about 30 mg to 180 mg per day. Generally, in the case of oral administration, an approximate daily dose of oxycodone in an opioid-naïve adult human may be from about 5 mg to about 200 mg, from about 10 mg to about 150 mg, or from about 20 mg to 100 mg per day, which is estimated for a patient of approximately 75 kg in weight.

Depending on the specific neuropathic condition being treated, the active compounds may be formulated and administered systemically, topically or locally. Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. For injection, the therapeutic agents of the invention may be formulated in aqueous solutions, suitably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

Alternatively, the compositions of the invention can be formulated for local or topical administration. In this instance, the subject compositions may be formulated in any suitable manner, including, but not limited to, creams, gels, oils, ointments, solutions and suppositories. Such topical compositions may include a penetration enhancer such as benzalkonium chloride, digitonin, dihydrocytochalasin B, capric acid, increasing pH from 7.0 to 8.0. Penetration enhancers which are directed to enhancing penetration of the active compounds through the epidermis are advantageous in this regard. Alternatively, the topical compositions may include liposomes in which the active compounds of the invention are encapsulated.

The compositions of this invention may be formulated for administration in the form of liquids, containing acceptable diluents (such as saline and sterile water), or may be in the form of lotions, creams or gels containing acceptable diluents or carriers to impart the desired texture, consistency, viscosity and appearance. Acceptable diluents and carriers are familiar to those skilled in the art and include, but are not restricted to, ethoxylated and nonethoxylated surfactants, fatty alcohols, fatty acids, hydrocarbon oils (such as palm oil, coconut oil, and mineral oil), cocoa butter waxes, silicon oils, pH balancers, cellulose derivatives, emulsifying agents such as non-ionic organic and inorganic bases, preserving agents, wax esters, steroid alcohols, triglyceride esters, phospholipids such as lecithin and cephalin, polyhydric alcohol esters, fatty alcohol esters, hydrophilic lanolin derivatives, and hydrophilic beeswax derivatives.

Alternatively, the active compounds of the present invention can be formulated readily using pharmaceutically acceptable carriers well known in the art into dosages suitable for oral administration, which is also preferred for the practice of the present invention. Such carriers enable the compounds of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatine, talc, calcium sulphate, vegetable oils, synthetic oils, polyols, alginic acid, phosphate buffered solutions, emulsifiers, isotonic saline, and pyrogen-free water.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilisers or agents that increase the solubility of the compounds to allow for the preparation of highly concentrated solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as., for example, maize starch, wheat starch, rice starch, potato starch, gelatine, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more therapeutic agents as described above with the carrier which constitutes one or more necessary ingredients. In general, the pharmaceutical compositions of the present invention may be manufactured in a manner that is itself known, e.g., by means of conventional mixing, dissolving, granulating, dragee-making, levigating, emulsifying, encapsulating, entrapping or lyophilising processes.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterise different combinations of active compound doses.

Pharmaceuticals which can be used orally include push-fit capsules made of gelatine, as well as soft, sealed capsules made of gelatine and a plasticiser, such as glycerol or sorbitol. The push-fit capsules can contain the active ingredients in admixture with filler such as lactose, binders such as starches, and/or lubricants such as talc or magnesium stearate and, optionally, stabilisers. In soft capsules, the active compounds may be dissolved or suspended in suitable liquids, such as fatty oils, liquid paraffin, or liquid polyethylene glycols. In addition, stabilisers may be added.

Dosage forms of the active compounds of the invention may also include injecting or implanting controlled releasing devices designed specifically for this purpose or other forms of implants modified to act additionally in this fashion. Controlled release of an active compound of the invention may be achieved by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be achieved by using other polymer matrices, liposomes and/or microspheres.

The active compounds of the invention may be administered over a period of hours, days, wks, or months, depending on several factors, including the severity of the neuropathic condition being treated, whether a recurrence of the condition is considered likely, etc. The administration may be constant, e.g., constant infusion over a period of hours, days, wks, months, etc. Alternatively, the administration may be intermittent, e.g., active compounds may be administered once a day over a period of days, once an hour over a period of hours, or any other such schedule as deemed suitable.

The compositions of the present invention may also be administered to the respiratory tract as a nasal or pulmonary inhalation aerosol or solution for a nebuliser, or as a microfine powder for insufflation, alone or in combination with an inert carrier such as lactose, or with other pharmaceutically acceptable excipients. In such a case, the particles of the formulation may advantageously have diameters of less than 50 micrometers, suitably less than 10 micrometers.

In order that the invention may be readily understood and put into practical effect, particular preferred embodiments will now be described by way of the following non-limiting examples.

### EXAMPLES

### Example 1

Assessment of Temporal Antinociceptive Potency of μ-Opioid Receptor Agonists in STZ Diabetic Rats

Materials and Methods

Jugular Vein Cannulation and Diabetes Induction

Deep and stable anaesthesia was induced with a mixture of ketamine (100 mg/kg, i.p.) and xylazine (16 mg/kg, i.p.) to facilitate insertion of a polyethylene cannula (previously filled with 0.1 ml of sterile saline) into the right common jugular vein. Jugular vein cannulae were tested for correct placement by the withdrawal of a small amount of blood. Diabetes was induced following an acute i.v. injection of streptozotocin (STZ) (85 mg/kg) in 0.1 M citrate buffer (pH 4.5) into the jugular vein.

Diabetes was confirmed by monitoring the water intake and blood glucose concentration in individual rats. For the acute study, blood glucose was monitored using either (Glucostix™) or a Precision QID™ test kit.

Consistent with the accepted standard protocol in the art, rats that drank greater than 100 ml of water per day by 7 days post-STZ injection, were classified as diabetic, and only rats with blood glucose concentrations exceeding 15 mM were included in the subsequent experiments. By comparison, the water intake of control non-diabetic rats was approximately 20 mL per day and blood glucose concentrations were in the range 5-6 mM, consistent with the previous studies well known in the art. The overall success rate for the induction of diabetes in the various experimental cohorts, was approximately 75%. Naïve non-diabetic rats (n=36) were used in the control experiments. Following STZ administration, benzylpenicillin (60 mg, s.c.) was administered to prevent infection and rats were monitored closely during surgical recovery. Rats were then housed singly or in pairs for period of 3 wks to 38 wks, depending upon the study cohort to which they belonged.

Drug Dosing Solutions

Stock solutions of morphine and oxycodone for s.c. administration were prepared by dissolving morphine hydrochloride or oxycodone hydrochloride in sterile saline to produce concentrations of 45 and 80 mg/ml (as the free base), respectively. Multiple aliquots of these stock solutions were stored at −20° C. until required. After thawing, aliquots of morphine or oxycodone stock solutions were serially diluted with sterile saline to produce the required opioid drug concentration for s.c. administration. Whilst under light anaesthesia with CO₂/O₂ (50:50%), rats received a single s.c. injection (100 μL) of one opioid or vehicle (saline) into the dorsal region of the base of the neck, using a 250 μL Hamilton syringe.

Assessment of Antinociception

Mechanical allodynia, the distinguishing feature of diabetic neuropathic pain, was quantified using von Frey filaments. Rats were placed in a metabolic cage (20 cm×20 cm×20 cm) with a metal mesh floor and allowed to acclimatise for approximately 10 min. von Frey filaments were used to quantify the lowest mechanical threshold required for a brisk paw withdrawal reflex. The force was applied to the plantar surface of the left hindpaw and held until the filament buckled slightly. The absence of a response after 5 s prompted application of the next filament of increasing force. Filaments available for use included those that produced a buckling weight of 2, 4, 5, 6, 8, 10, 12, 14, 16, and 18 g. Filaments were calibrated daily before undertaking antinociceptive testing. A score of 20 g was given to animals that did not respond to light pressure applied to the plantar surface of the left hindpaw by any of the von Frey filaments. Pre-drug (opioid or saline) responses were the mean of three readings taken ≈5 min apart. Assessment of von Frey filament responsiveness was determined at the following times post-opioid (or saline) administration: 15, 30, 45, 60, 90, 120 and 180 min.

Data Analysis

The von Frey scores for individual rats were converted to the Percentage of the Maximum Possible Antinociceptive Effect (% MPE), according to the formula:

$$\% MPE = \frac{( Post Drug Threshold \text{-} Predrug Threshold )}{( Maximum threshold \text{-} Predrug Threshold )} \times \frac{100}{1}$$

$$where maximum VFF threshold = 20 g$$

The area under the % MPE versus time curve from time=0-180 min (% MPE AUC) was calculated using the trapezoidal rule. The mean (±SEM) percentage maximum AUC (% Max AUC) was calculated according to the following formula:

$$\% Max AUC = \frac{\% MPE AUC}{MAXIMUM \% MPE AUC} \times \frac{100}{1}$$

$$where maximum \% MPE AUC = 263 \% MPE \text{-} h$$

The % Max AUC for each morphine or oxycodone dose was plotted versus the respective drug dose to produce individual dose-response curves. ED₅₀ doses (mean±SEM) for morphine or oxycodone were estimated using non-linear regression of the % Max AUC versus log dose values as implemented in the statistical analysis package, GraphPad Prism™. ED₅₀ estimation was facilitated by the inclusion of theoretical maximum and minimum % Max AUC values.

Study Design and Opioid Dosing Regimens

This study comprised three groups of STZ-diabetic DA rats. At 3 wks post-STZ administration, Group 1 (n=36, 207±5 g, mean±SEM) rats received one of three bolus doses of s.c. morphine or oxycodone. Initial doses of s.c. oxycodone and morphine were those that had been used previously in our laboratory to alleviate tactile allodynia secondary to a chronic constriction injury of the sciatic nerve. Subsequent doses were chosen to facilitate construction of dose-response curves for the alleviation of tactile allodynia. Antinociception was quantified using calibrated von Frey filaments.

By contrast, the acute antinociceptive responses of Group 2 diabetic rats (n=25, 256±3.6 g, mean±SEM) were studied over a 9 wk period such that individual rats received one of three bolus doses of s.c. morphine or oxycodone to produce dose-response curves at 9 wks post-STZ administration.

Group 3 diabetic rats (n=37, 233.0±5.1 g, mean±SEM) were studied serially over a six month study period such that individual rats received one of three bolus doses of s.c. morphine or oxycodone to produce dose-response curves at 12 and 24 wks post-STZ administration.

At each time of antinociceptive testing, rats in each experimental group were randomly assigned to receive bolus sc doses of either oxycodone or morphine such that each rat received two or three doses of opioid with four complete days of washout between doses.

Additionally, a group of naïve, weight-matched control non-diabetic rats (n=36, 210±4 g, mean±SEM) were studied such that individual rats received one of three bolus doses of s.c. morphine or oxycodone to produce antinociceptive dose-response curves.

Results

Diabetic Neuropathic Pain

Development of Diabetic Neuropathic Pain in Short-Term (3 wks) and Long-Term (6 mths) Studies

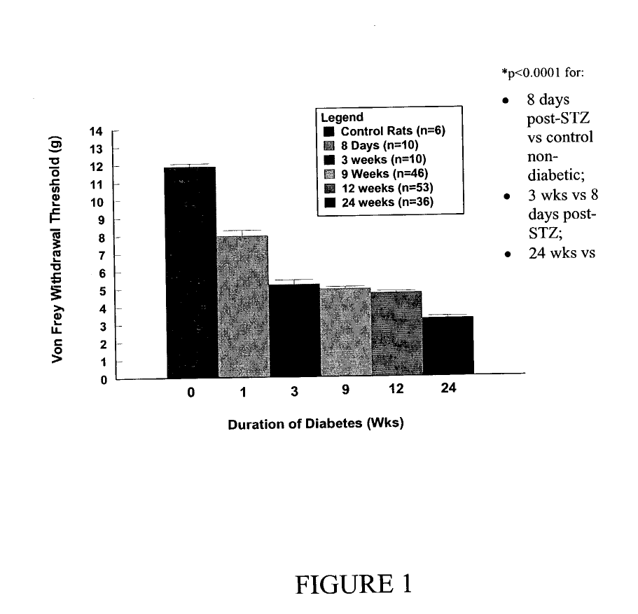

By day 8 post-STZ injection, there was a significant (p<0.0001) reduction in the mean (±SEM) paw withdrawal threshold from 11.9 (±0.15) g in control non-diabetic rats to 8.0 (±0.3) g (FIG. 1). By 3-wks post-STZ, there was a further significant (p<0.0001) decrease in the mean (±SEM) paw withdrawal threshold to 5.23 (±0.34) g (FIG. 1). For the next 2 mths, the mean (±SEM) baseline paw withdrawal thresholds were relatively stable with values being 5.0 (±0.1) g and 4.7 (±0.1) g at 9 and 12 wks post-STZ, respectively. There was a further small but significant (p<0.0001) decrease in baseline paw withdrawal thresholds between 12 and 24 wks such that at 24 wks post-STZ the mean (±SEM) paw withdrawal threshold was 3.3 (±0.1) g (FIG. 1). Taken together, these data show the development and maintenance of mechanical allodynia (defining symptom of PDN) for the 6-month study period in rats with STZ-induced diabetes.

Longitudinal Study of the Effects of STZ-Diabetes on the Potency of Morphine and Oxycodone for the Relief of Mechanical Allodynia

3 wks Post-STZ Injection

Following s.c. administration of morphine (4 mg/kg), peak antinociception (70% MPE) was evoked within 15 min. Thereafter, levels of antinociception declined to baseline levels (<20% MPE) by 90 min post-dosing. Following bolus s.c. oxycodone administration (1.7 mg/kg), peak antinociception (90% MPE) was produced by 30 min post-injection which then declined to baseline levels (<20% MPE) by 120 min post-dosing. Similar profiles for the degree of antinociception (% MPE) versus time were produced by the other bolus doses of s.c. oxycodone and morphine administered. The corresponding mean (±SEM) ED₅₀ doses for morphine and oxycodone in diabetic rats were 6.1 (±0.3) mg/kg and 2.0 (±0.15) mg/kg (Table 1), respectively, indicating that oxycodone is ≈3 times more potent than morphine for the alleviation of mechanical allodynia in STZ-diabetic rats. By comparison, in the absence of diabetes (naïve control rats), the ED₅₀ doses for morphine and oxycodone were 2.4 (±0.3) mg/kg and 1.2 (±0.04) mg/kg respectively (Table 1). Taken together, these data show that STZ-induced diabetes in DA rats produced an ≈2.5-fold rightward shift in the dose-response curve for morphine (p<0.05) and an ≈1.7-fold shift for oxycodone (p<0.05) by 3 wks post-STZ administration.

9 wks Post-STZ Injection

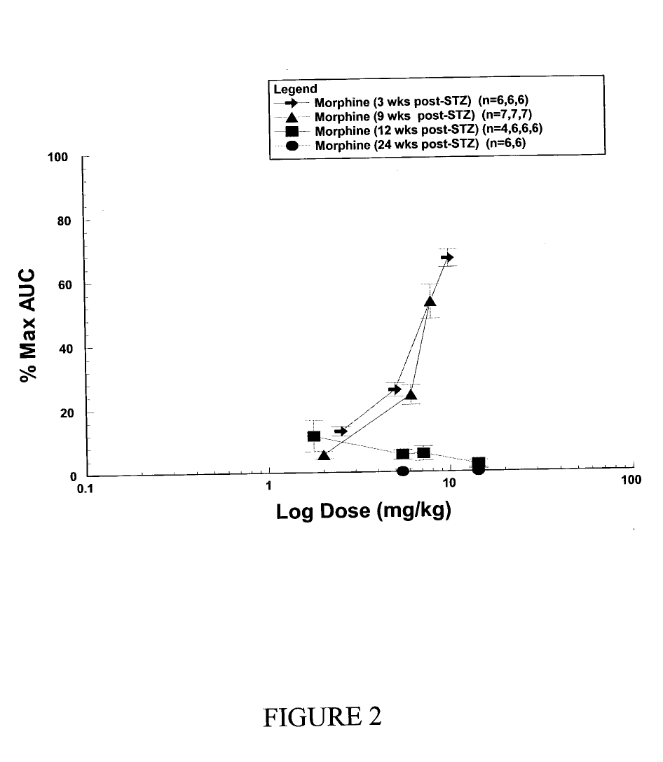

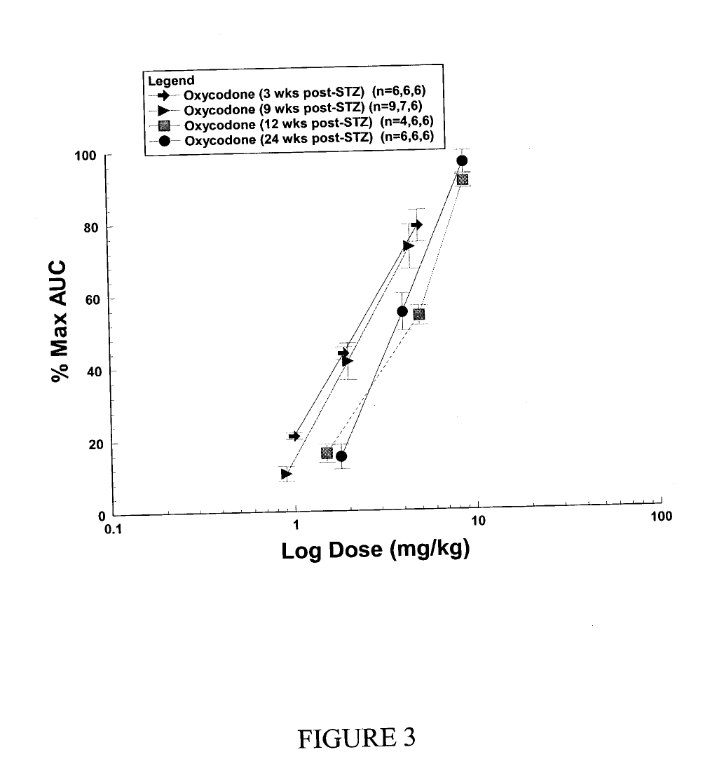

The mean (±SEM) antinociceptive response (% MPE) versus time curves and the log dose-response curves for s.c. morphine and oxycodone are shown in FIGS. 2 and 3. For both s.c. morphine and s.c. oxycodone, the 9 wk dose-response curves were not significantly different from the respective 3 wk dose-response curves. Specifically the mean (±SEM) ED₅₀ values for morphine and oxycodone were 6.1 (±0.4) mg/kg and 2.1 (±0.4) mg/kg, respectively.

12 wks Post-STZ Injection

Remarkably, at 12 wks post-STZ administration, the antinociceptive potency of morphine was completely abolished. To test whether higher doses of morphine would elicit an antinociceptive response, a dose ≈2.5 times the original ED₅₀ value (14.2 mg/kg) was given. However, morphine efficacy remained completely abolished. Initial trials of higher s.c. morphine doses (18 mg/kg) produced mild neuro-excitatory behaviours (myoclonus and biting of bottom of wire mesh cage) together with an absence of antinociception in these STZ-diabetic rats, and so no further escalation of the morphine dose was undertaken.

By contrast, the antinociceptive efficacy of oxycodone was maintained at 12 wks post-STZ injection although there was a further 2-fold decrease in potency between 9 and 12 wks. Specifically, the mean (±SEM) ED₅₀ dose for oxycodone in these 12 wk post-STZ diabetic rats was 4.1 (±0.3) mg/kg (Table 1) c.f. 2.1 (±0.4) mg/kg at 9 wks, for the alleviation of mechanical allodynia.

24 wks Post-STZ Injection

In a manner analogous to that observed at 12 wks post-STZ administration, the efficacy of morphine remained completely abolished, i.e. there was no temporal reversal of the loss of morphine antinociceptive efficacy. By contrast, the potency of oxycodone was found to be the same as that determined at 12 wks post-STZ (ED₅₀=4.2 (±0.3) mg/kg).

Summary

The above experiments have shown that for both oxycodone and morphine there were temporal stepwise decreases in antinociceptive potency, and that the time course for this loss of potency differed between the two opioids. Consistent with clinical opinion that morphine is ineffective for the relief of PDN in patients, these results show that efficacy of morphine for the alleviation of mechanical allodynia in diabetic rats was completely abolished by 12 wks post-STZ administration. By contrast, the efficacy of oxycodone was maintained for the full 24 wk study period, albeit with a 4-fold decrease in antinociceptive potency at 12 wks which remained unchanged at 24 wks relative to control non-diabetic rats.

Importantly, the antinociceptive efficacy of oxycodone was maintained throughout the 24 wk post-STZ study period, albeit with a 4-fold decrease in the ED₅₀ relative to naïve non-diabetic rats. Extrapolated to the clinical setting, this finding indicates that oxycodone (in contrast to morphine) retains efficacy for relief of painful diabetic neuropathy in patients. Watson et al (Neurology, 50 1837-41 1998) showed that oxycodone was effective for the relief of neuropathic pain in patients with post-herpetic neuralgia, a difficult to treat persistent pain state.

Oxycodone was found to be ≈2-fold more potent than morphine at 3 and 9 wks post-STZ when given by the s.c. route to naïve non-diabetic rats. Additionally, previous studies in the laboratory of the inventors have shown that s.c. oxycodone is ≈3 times more potent than morphine when quantified using the tail flick test in naïve Dark Agouti rats and ≈4 times more potent than morphine for the relief of mechanical allodynia in rats with a chronic constriction injury (CCI) of the sciatic nerve (Smith et al., 2001, Eur J Pain 5 (Suppl A): 135-136).

### Example 2

L-Arginine Restores the Antinociceptive Potency of Opioid-Receptor Agonists in PDN

Materials and Methods

Study Design, L-Arginine Administration and Opioid Dosing Regimens:

This study comprised three groups of STZ-diabetic DA rats: STZ-diabetic DA rats in Group 1 (n=25, 256±3.6 g, mean±SEM) were studied serially over a 6-month period such that individual rats received (i) one of three bolus s.c. doses of either morphine or oxycodone to produce dose-response curves at 9, 12 and 24 wks post-STZ administration, or (ii) an ED₅₀ dose of morphine and/or oxycodone at 16 and 20 wks post-STZ administration. For each testing session, rats received single s.c. doses of either morphine or oxycodone on two or three occasions in a cross-over design, with four complete days of washout between doses. At 9 wks post-STZ administration, Group 1 STZ-diabetic rats received a dietary intervention of an L-arginine supplement (1 g per day) incorporated into rat chow, until the end of the 24 wk study period.

Group 2 STZ-diabetic rats (n=17, 233.7±4.1 g, mean±SEM) were studied serially over a 6-month period such that individual (n=6) rats received the ED₅₀ dose of either s.c. morphine and/or s.c. oxycodone (6.1 mg/kg or 2.0 mg/kg, respectively) to evaluate the acute antinociceptive responses at 14, 18, and 22 wks post-STZ administration. At 14 wks post-STZ administration, a dietary intervention comprising L-arginine supplementation (1 g per day in rat chow) was initiated which was continued for another 8 wks.

Group 3 STZ-diabetic rats, (n=6, 224.7±2.9 g, mean±SEM) were the same rats used in Example 1 above. These rats had previously received single s.c. bolus doses of oxycodone or morphine at 9, 12, and 24 wks post-STZ. Thereafter, individual rats received the ED₅₀ dose of either s.c. morphine or s.c. oxycodone to produce acute antinociceptive response versus time curves at 34 and 38 wks. At 30 wks post-STZ administration, dietary L-arginine supplementation (1 g/day in rat chow) was initiated and continued for 8 wks.

Additionally, a group of weight-matched naïve control non-diabetic DA rats (n=18, 236.8±2.5 g, mean±SEM) were studied such that individual rats received one of three doses of either s.c. morphine or s.c. oxycodone to produce antinociceptive dose-response curves. Weight-matched naïve control DA rats received dietary L-arginine supplementation (1 g per day in rat chow) for at least 1 wk prior to acute opioid administration and concomitant antinociceptive testing. Importantly, since diabetic rats eat twice as much as naïve control non-diabetic rats, the concentration of L-arginine in rat chow administered to control non-diabetic rats was doubled to maintain consistent L-arginine dosing between the STZ-diabetic rats and the control non-diabetic rats.

Results

Diabetic Neuropathic Pain

Long-term Studies of the Development of Diabetic Neuropathic Pain and the Effects of L-Arginine Supplementation on von Frey Paw Withdrawal Thresholds

The mean (±SEM) paw withdrawal thresholds found in this cohort of drug-naïve STZ-diabetic DA rats were significantly lower (p<0.0001) than the respective mean (±SEM) paw withdrawal threshold found in control non-diabetic rats (11.9±0.2 g). Specifically, the mean (±SEM) paw withdrawal threshold decreased significantly (p<0.0001) from 11.9 (±0.2) g in non-diabetic rats to 6.8 (±0.3) g by 9 wks post-STZ (Group 1). Similarly, the significant (p<0.0001) decrease in the mean (±SEM) paw withdrawal thresholds observed in Group 2 STZ-diabetic rats at 14 wks post-STZ (3.8±0.2 g) and in Group 3 STZ-diabetic rats at 24 wks post-STZ (3.1±0.3 g) relative to that for naïve control non-diabetic rats (11.9±0.2 g), indicated the development and maintenance of tactile allodynia (defining symptom of PDN) for up to 6-mths following the induction of diabetes in rats. These findings show the reproducibility of the induction and maintenance of STZ-diabetes and the associated tactile allodynia, in our laboratory.

Group 1

Dietary administration of L-arginine to STZ-diabetic rats in Group 1 for 15 wks (from 9 to 24 wks post-STZ) resulted in paw withdrawal thresholds of 6.8 (±0.3) g at 9 wks post-STZ, 4.3 (±0.1) g at 12 wks post-STZ, which increased marginally to 5.2 (±0.1) g at 24 wks post-STZ.

Group 2

Similarly, initiation of dietary supplementation of L-arginine to Group 2 STZ-diabetic rats at 14 wks post-STZ administration resulted in small increases in the paw withdrawal thresholds from 3.8 (±0.2) g at 14 wks post-STZ to 4.9 (±0.2) g and 6.1 (±0.4) g after 4 (18 wks post-STZ) and 8 wks (22 wks post-STZ) of L-arginine treatment, respectively.

Group 3

Although dietary supplementation with L-arginine did not commence in Group 3 STZ-diabetic rats until 30 wks post-STZ administration, small but significant increases in paw withdrawal thresholds were observed such that the values increased from 3.1 (±0.3) g at 24 wks, to 3.9 (±0.2) g and 5.0 (±0.2) g at 4 wks and 8 wks after the initiation of L-arginine treatment (34 and 38 wks post-STZ, respectively). These data taken together, are consistent with the development and maintenance of tactile allodynia (defining symptom of PDN) for the entire experimental period in rats administered STZ.

Control Group with L-Arginine

Dietary administration of L-arginine to weight-matched control non-diabetic rats for 1 wk had no significant effect on baseline paw withdrawal thresholds (13.3±0.12 g), relative to the values found in control non-diabetic rats that received a standard rat chow diet (11.9±0.2 g).

Effect of Dietary L-Arginine Supplementation on Body Weight in STZ-Diabetic Rats

Group 1

Just prior to STZ administration, Group 1 rats weighed 256.0 (±3.6) g. Consistent with previous investigations in the laboratory of the inventors, STZ administration resulted in an approximate 10% decrease in body weight such that STZ-diabetic rats weighed 223.6 (±5.5) g by 9 wks post-STZ. After 3 and 7 wks of dietary L-arginine supplementation (12 wks and 16 wks post-STZ administration, respectively) mean (±SEM) weights remained relatively stable at 229.0 (±6.0) g and 218.0 (±7.2) g, respectively. It was found that after 11 and 15 wks of L-arginine treatment (20 and 24 wks post-STZ administration, respectively) the mean (±SEM) body weights were 253.4 (±9.9) g at 20 wks (n=6) and 234.5 (±5.1) g at 24 wks (n=25) post-STZ. The approximate 5% difference in mean body weight between rats at 11 and 15 wks following initiation of L-arginine supplementation (20 and 24 wks post-STZ administration) is almost certainly due to the significant difference in sample size between the 2 groups. Importantly, body weight was maintained throughout an extended period of L-arginine treatment (15 wks) with a gradual increase in body weight being found after approximately 10 wks of dietary L-arginine supplementation.

Group 2

For Group 2 STZ-diabetic rats, the mean (±SEM) weight at the time of STZ administration was 239.7 (±4.9) g which again decreased by ≈10% to 211.5 (±3.4) g at 14 wks post-STZ. After 4 and 8 wks of dietary L-arginine supplementation (18 and 22 wks post-STZ), the mean (±SEM) weights of the diabetic rats remained relatively stable at 203.2 (±6.4) g and 220.9 (±11.6) g, respectively.

Group 3

The mean (±SEM) weight of Group 3 rats at the time of STZ administration was 228.8±(4.18) g. The mean weight of these rats decreased by approximately 10% to 201.0 (±7.1) g which was maintained until 24 wks post-STZ administration. By 4 wks (34 wks post-STZ) after the initiation of the dietary L-arginine intervention the mean (±SEM) weight of these rats was 207.8 (±10.7) g. Consistent with STZ-diabetic rats in Group 2 that also received a dietary L-arginine intervention for 8 wks, the mean (±SEM) weight of these rats increased by a small but significant (p<0.05) extent between 4 and 8 wks after initiation of the dietary L-arginine supplement reaching 221.7 (±11.7) g by 8 wks of treatment (i.e. 38 wks post-STZ).

Control Group with L-Arginine

The mean (±SEM) weight of weight-matched control non-diabetic rats given dietary L-arginine supplementation for 1 wk prior to antinociceptive testing increased from 215.2 (±2.0, n=8) g to 236.3 (±2.5, n=18) g, as expected for non-diabetic control rats of this age.

Longitudinal Study of the Effects of a Dietary L-Arginine Intervention in Rats with STZ-Diabetes on the Potency of Morphine and Oxycodone for the Relief of Mechanical Allodynia

Control Rats with L-Arginine

Statistical comparison of the dose-response curve for s.c. morphine in control opioid-naïve, non-diabetic rats administered the dietary L-arginine intervention for 1 wk prior to antinociceptive testing, indicates that the ED₅₀ for morphine does not differ significantly (p>0.05) from that determined in control rats that received a standard rat chow diet. Similarly, the ED₅₀ value for oxycodone in rats that received the dietary L-arginine intervention (1.0±0.1 mg/kg) was not significantly (p>0.05) different from that for rats fed a standard rat chow diet (1.2±0.1 mg/kg). These findings show that chronic administration of L-arginine did not modulate the antinociceptive actions of oxycodone in a manner analogous to morphine in opioid-naïve non-diabetic control rats.

Group 1 STZ-Diabetic Rats Administered Dietary L-Arginine Supplementation

9 wks Post-STZ Injection—Prior to Initiation of Dietary L-Arginine Supplementation

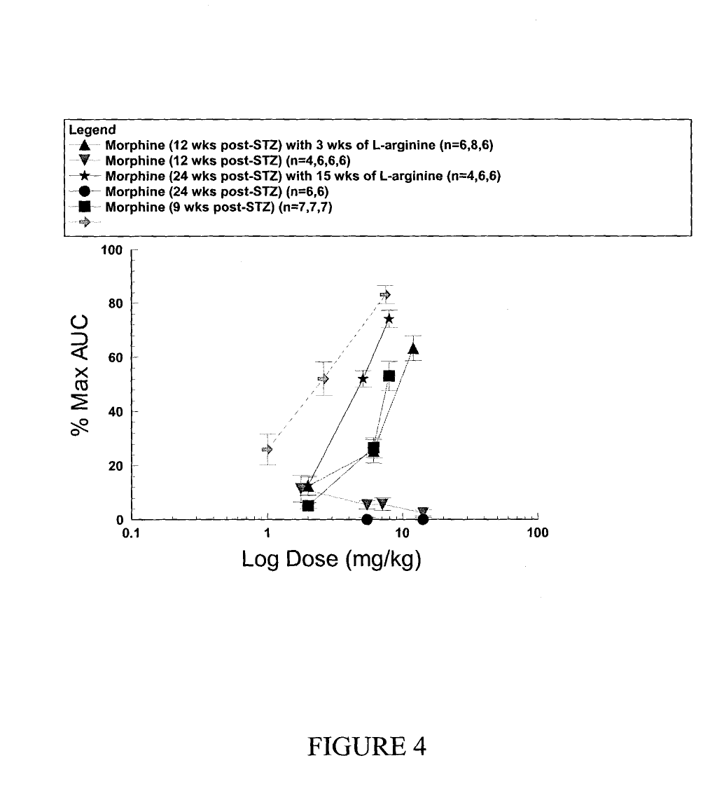

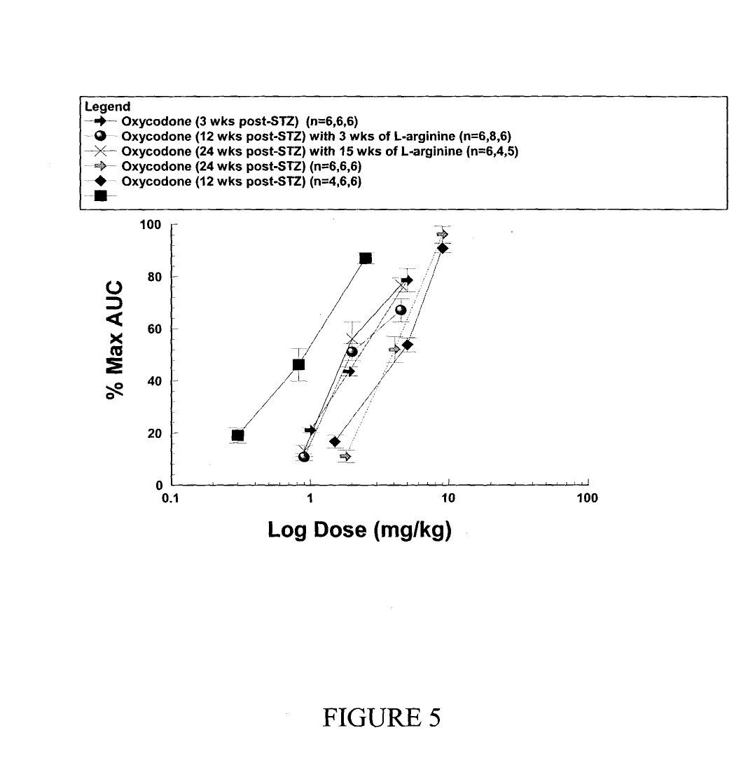

The dose-response curves for both s.c. morphine and s.c. oxycodone at 9 wks post-STZ administration (FIG. 4 and FIG. 5) were not significantly different from the comparable dose-response curves determined at 3 wks post-STZ administration in earlier studies in the laboratory of the inventors. Specifically the mean (±SEM) ED₅₀ values for morphine and oxycodone were 6.1 (±0.3) mg/kg and 2.1 (±0.4) mg/kg, respectively.

12 wks Post-STZ—After 3 wks of Dietary L-Arginine Supplementation

Unexpectedly, 3 wks of dietary L-arginine supplementation prevented the abolition of morphine's antinociceptive efficacy that occurred between 9 and 12 wks post-STZ administration in diabetic rats fed a standard rat chow diet such that the (±SEM) morphine ED₅₀ (7.0±0.5 mg/kg) was found to be not significantly different (p>0.05) from that determined in STZ-diabetic rats fed a standard rat chow diet at 3 and 9 wks post-STZ administration (6.1±0.3 mg/kg) (FIG. 4).

Similarly, the antinociceptive potency of oxycodone in this same group of rats was also maintained such that the oxycodone ED₅₀ was identical (2.0±0.3 mg/kg) (FIG. 5) to that established earlier by the inventors for diabetic rats at 3 and 9 wks post-STZ administration (2.1±0.4 mg/kg). Thus, 3 wks of dietary L-arginine supplementation prevented the 2-fold decrease in oxycodone potency that occurred between 9 and 12 wks post-STZ administration in diabetic rats fed a standard rat chow diet.

16 wks Post-STZ Injection—After 7 wks of Dietary L-Arginine Supplementation

Administration of the ED₅₀ dose of morphine (6.1 mg/kg, determined at 3 and 9 wks post-STZ administration) to diabetic rats that had received 7 wks of dietary L-arginine supplementation (16 wks post-STZ) showed that the efficacy of morphine for the relief of mechanical allodynia was maintained. Specifically, following acute s.c. administration of this ED₅₀ dose of morphine (6.1 mg/kg), the % MPE AUC (±SEM) value was 101.9 (±1.9) % MPE-h which was significantly (p<0.05) larger than the respective % MPE AUC value (63.4±7.5% MPE-h) found in diabetic rats fed a standard rat chow diet at 9 wks post-STZ. These findings show that 7 wks of dietary L-arginine supplementation increased the potency of s.c. morphine towards that found in weight-matched control non-diabetic rats.

20 wks Post-STZ Injection—11 wks of Dietary L-Arginine Supplementation

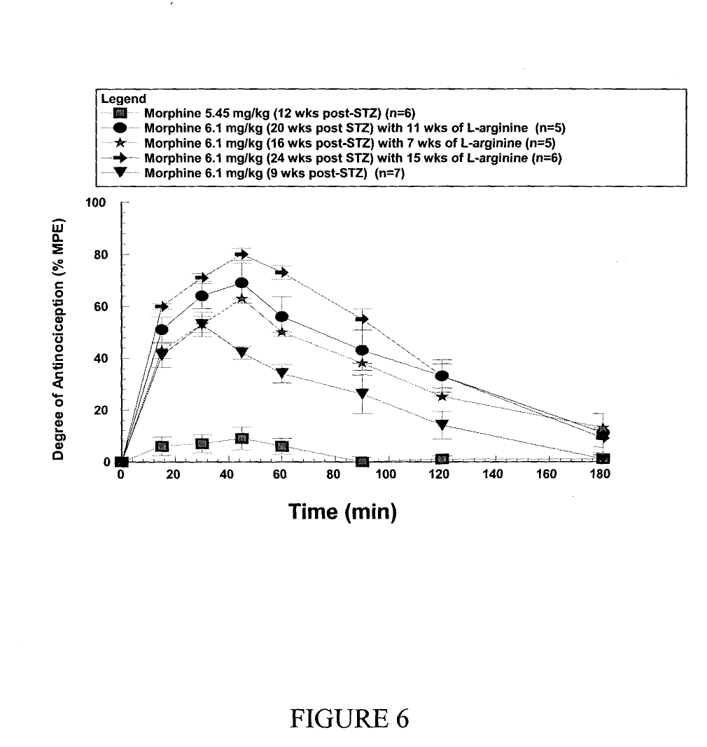

After 11 wks of dietary L-arginine supplementation the % MPE AUC (±SEM) evoked by single s.c. doses of the morphine ED₅₀ (6.1 mg/kg, 3 & 9 wks post-STZ) increased from 63.4±7.5% MPE-h in diabetic rats fed a standard rat chow diet to 119.2 (±19.1) % MPE-h in diabetic rats fed rat chow containing the L-arginine supplement. These data show that dietary L-arginine supplementation in STZ-diabetic rats increased the potency of morphine for the relief of mechanical allodynia to ≈90% of that found in control non-diabetic rats (% MPE AUC=136.9±16.1% MPE-h) (FIG. 6).

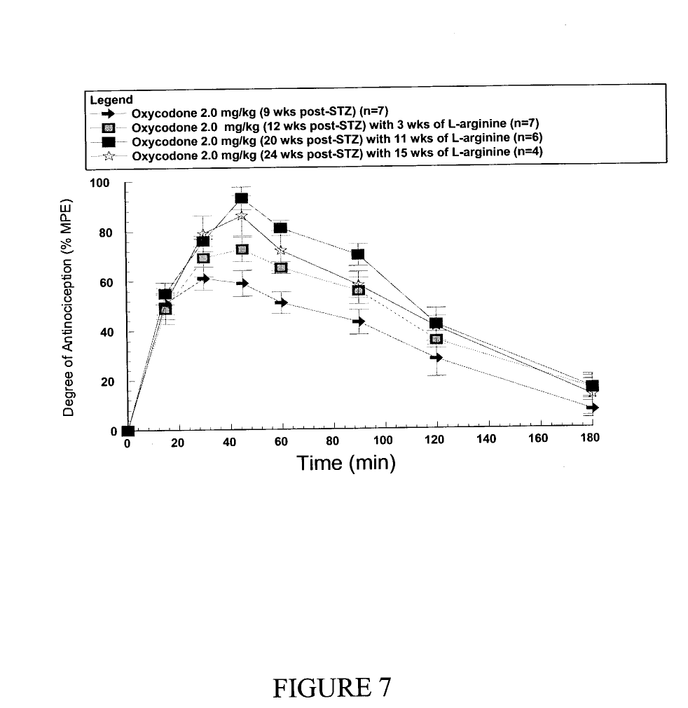

Additionally, the extent and duration of antinociception (% MPE AUC (±SEM)) evoked by acute, s.c. administration of the ED₅₀ dose of oxycodone (2.0 mg/kg) to these same rats that received 11 wks of dietary L-arginine supplementation, was significantly (p<0.05) increased (160.3±7.6% MPE-h) relative to the % MPE AUC value (108.7±13.2% MPE-h) evoked by the same dose of s.c. oxycodone in diabetic rats fed standard rat chow at 9 wks post-STZ administration (FIG. 7). These findings show that dietary L-arginine supplementation increased the potency of oxycodone for the relief of mechanical allodynia to ≈150% of that found in diabetic rats fed standard rat chow diet at 9 wks post-STZ.

24 wks Post-STZ Injection—15 wks of Dietary L-Arginine Intervention

In a manner analogous to that observed for STZ-diabetic rats that received 3, 7 and 11 wks of dietary L-arginine supplementation, the efficacy of morphine was maintained (FIG. 4), i.e. the abolition of morphine efficacy observed in 24 wk STZ-diabetic rats fed a standard rat chow diet was prevented and the potency of morphine was increased relative to that determined after 11 wks of the dietary L-arginine intervention. This is exemplified by the apparent leftward shift in the dose-response curve for s.c. morphine (FIG. 4) such that the ED₅₀ value (5.0±0.9 mg/kg) was less than that determined in 9 wk STZ-diabetic rats (6.1±0.4 mg/kg). However, the ED₅₀ was still approximately twice that determined (2.4 (±0.7) mg/kg) in naïve non-diabetic rats.

By contrast, 15 wks of dietary L-arginine supplementation of STZ diabetic rats (24 wks post-STZ) maintained the potency of oxycodone (ED₅₀=1.8±0.3 mg/kg) (FIG. 5) at approximately the same as that determined at 9 wks post-STZ (2.1±0.4 mg/kg).

Group 2 STZ-Diabetic Rats: Effect of an 8 wk Dietary L-Arginine Intervention on the Potency of Morphine and Oxycodone for the Relief of Mechanical Allodynia

14 wks Post-STZ—Just Prior to Initiation of the Dietary L-Arginine Intervention

Administration of the 3 and 9 wk post-STZ ED₅₀ dose of s.c. morphine (6.1 mg/kg) to diabetic rats at 14 wks post-STZ administration, revealed that the antinociceptive efficacy of acutely administered s.c. morphine was completely abolished.

18 wks Post-STZ Injection—4 wks of Dietary L-Arginine Intervention

Remarkably, 4 wks of dietary L-arginine supplementation in Group 2 diabetic rats (18 wks post-STZ) restored the antinociceptive efficacy of s.c. morphine (6.1 mg/kg) such that the extent and duration of antinociception (% MPE AUC values) was 109.8±28.6% MPE-h which represents a 21-fold increase in the extent and duration of morphine antinociception relative to the respective antinociceptive response (AUC value) evoked by the same dose of morphine in 14-wk STZ-diabetic rats fed a standard rat chow diet (5.2±2.5% MPE-h).

22 wks Post-STZ Injection—8 wks of Dietary L-Arginine Intervention

Administration of this same dose of s.c. morphine (6.1 mg/kg) to STZ-diabetic rats that had received dietary L-arginine supplementation for 8 wks (22 wks post-STZ) evoked a further increase in the extent and duration of morphine's antinociceptive effects such that the % MPE AUC value was 149.5±9.5% MPE-h which was significantly larger (p<0.05) than that observed after only 4 wks of dietary L-arginine supplementation and not significantly different (p<0.05) from the antinociceptive response found in naïve non-diabetic control rats (136.9±16.1% MPE-h). In these same rats (8 wks dietary L-arginine intervention) the extent and duration of antinociception (% MPE AUC) evoked by oxycodone in a dose of 2.0 mg/kg (ED₅₀ in 9 wk STZ-diabetic rats) was significantly (p<0.05) larger (139.4±9.4 MPE-h) than that found in 12-wk STZ-diabetic rats fed a standard rat chow diet (37.0±1.1% MPE-h).

Group 3 STZ-Diabetic Rats—Effects of an 8 wk Dietary L-Arginine Intervention on the Potency of Morphine and Oxycodone for the Relief of Mechanical Allodynia

24 wks Post-STZ: No L-Arginine Treatment

At 24 wks post-STZ, the efficacy of morphine remained completely abolished. Additionally, the potency of oxycodone was found to be the same as that determined in diabetic rats at 12 wks post-STZ administration in earlier studies (ED₅₀=4.2 (±0.3) mg/kg).

34 wks Post-STZ Injection—4 wks of Dietary L-Arginine Intervention

Remarkably, 4 wks of dietary L-arginine supplementation (from 30 to 34 wks post-STZ) partially restored the antinociceptive potency of morphine despite the fact that morphine's antinociceptive efficacy had been abolished since 12 wks post-STZ administration. Specifically, the extent and duration of antinociception evoked by a single s.c. dose bolus dose of morphine (6.1 mg/kg, ED₅₀ at 3 and 9 wks post-STZ) was 62.2±15.8% MPE-h which was almost identical to the % MPE AUC value (63.4±7.5% MPE-h) determined in diabetic rats fed a standard rat chow diet at 9 wks post-STZ.

38 wks Post-STZ Injection: 8 wks of Dietary L-arginine

Extension of the dietary L-arginine intervention from 4 to 8 wks (30 to 38 wks post-STZ administration) resulted in a further restoration of morphine's antinociceptive potency. Specifically, the % MPE AUC evoked by a single bolus dose of s.c. morphine (6.1 mg/kg) was 117.1 (±15.4) % MPE-h which was approximately 190% larger than the respective % MPE AUC values found after only 4 wks of the L-arginine dietary supplement (62.2±15.8% MPE-h).

In the same rats given an 8 wk dietary L-arginine intervention, administration of a single bolus dose of s.c. oxycodone (2.0 mg/kg, ED₅₀ at 38 wks post-STZ) evoked a similar extent and duration of antinociception (% MPE AUC=147.0±1.9% MPE-h) to that evoked by oxycodone in a dose of 4.0 mg/kg in 24 wk post-STZ diabetic rats fed on a standard rat chow diet (144.0±13.7 MPE-h). These data indicate that 8 wks of dietary L-arginine supplementation restored the potency of oxycodone to match that determined in STZ-diabetic rats at 3 and 9 wks post-STZ in rats fed a standard rat chow diet.

Summary

The potency of oxycodone and morphine in rats with mechanical allodynia (defining symptom of PDN) secondary to the induction of diabetes, was decreased by ≈2-fold by 9 wks post-STZ, relative to that found in weight-matched, control non-diabetic rats. However, dietary supplementation with L-arginine from 9 to 12 wks post-STZ, prevented the abolition of morphine efficacy that was observed at 12 wks post-STZ in comparable diabetic rats fed a standard rat chow diet. Similarly, 3 wks of dietary L-arginine supplementation from 9 to 12 wks post-STZ, prevented the 2-fold decrease in oxycodone potency that was observed between 9 and 12 wks post-STZ in diabetic rats fed a standard rat chow diet. Additionally, not only was morphine efficacy maintained in diabetic rats given the L-arginine dietary supplement from 9-12 wks post-STZ, but similar to oxycodone, the potency of morphine for the relief of mechanical allodynia was not significantly different from that observed in 3 and 9 wks post-STZ diabetic rats.

Remarkably, initiation of the dietary L-arginine intervention after morphine efficacy had been completely abolished in diabetic rats (i.e., 14 and 30 wks post-STZ for groups 2 and 3, respectively), restored morphine efficacy after as little as 4 wks of the dietary L-arginine intervention. After 8 wks of the dietary L-arginine supplement, the potency of morphine was further increased such that the ED₅₀ was not significantly different from that found at 3 wks post-STZ in diabetic rats fed a standard rat chow diet. These findings for morphine were mirrored for oxycodone such that late initiation of the dietary L-arginine intervention (i.e. at 14 and 30 wks post-STZ) resulted in a reversal of the 2-fold decrease in the antinociceptive potency of oxycodone seen from 12 wks onwards in STZ-diabetic rats. These marked improvements in the potency of single s.c. doses of oxycodone and morphine following 4-8 wks of the dietary intervention, occurred despite there being no reversal of the underlying allodynic pain state in diabetic rats.

### Example 3

Preparation of Morphine-Nitric Oxide Conjugate 1

Morphine 1

Morphine hydrochloride trihydrate (1.5 g) was dissolved in the minimum amount of water (RO type) (˜20 mL) and to this was added enough saturated sodium hydrogen carbonate to precipitate morphine. Morphine 1 was collected by vacuum filtration and washed with distilled water (20 mL) followed by small amounts of cold diethyl ether (5 mL). The white solid, protected from light with aluminium foil, was placed under high vacuum (0.01 mmHg) for 3 h.

5-Nitratovaleric Acid 2

The titled compound was prepared following the procedure of EP 0 984 012 A2 (K. M. Lundy, M. T. Clark). Briefly, silver nitrate (23.48 g, 0.153 mol) was pre-dried under high vacuum (0.01 mmHg) and subsequently dissolved in anhydrous acetonitrile (70 mL) under an argon atmosphere. The solution was warmed to 50° C. and 5-bromovaleric acid (5 g, 0.028 mol) [dissolved in anhydrous acetonitrile (3 mL)] added quickly via syringe. A precipitate formed instantaneously. The mixture was then heated at 80° C. for 20 mins. On cooling the precipitate (AgBr) was removed by filtration. The filtrate was concentrated and the residue partitioned between ethyl acetate and water. The ethyl acetate layer was then washed with water, dried (Na₂SO₄), concentrated and further dried under vacuum (0.01 mm Hg). The titled compound was used without further purification.

Morphine NO Donor 3

Freshly prepared morphine 1 (500 mg, 1.75 mmol), dicyclohexylcarbodiimide (362 mg, 1.75 mmol), and 5-nitratovaleric acid 2 (286 mg, 1.75 mmol) were dissolved in anhydrous chloroform (90 mL) under an argon atmosphere. The mixture was refluxed for 12 h and allowed to cool. Additional dicyclohexylcarbodiimide (362 mg, 1.75 mmol), and 5-nitratovaleric acid (286 mg, 1.75 mmol) were added and refluxing continued for 6 h. On cooling the solvent was removed in vacuo and the residue dissolved in a solution of warmed ethyl acetate/methanol (6:4) (˜5 mL) and filtered to remove N,N-dicyclohexylurea. The filtrate is concentrated and subjected to column chromatography (ethyl acetate/methanol; 6:4) on silica gel which affords morphine derivative 3 as a pale yellow solid (600 mg, 80%). ¹H n.m.r (200 MHz) 1.70-1.95 (m, 5H), 2.07 (dt, 1H), 2.22-2.38 (m, 2H), 2.42 (s, 3H), 2.54-2.73 (m, 3H), 3.05 (d, 1H), 3.35 (bs, OH), 3.33-3.40 (m, 2H), 4.08-4.20 (m, 1H), 4.40-4.55 (m, 2H), 4.90 (d, 1H), 5.20-5.34 (m, 1H), 5.67-5.78 (m, 1H), 6.65 (dd, 2H). Mass spectrum m/z (EI) 430 (M^{+•}, 27%), 384 (1), 366 (1), 354 (18), 326 (1), 285 (100), 268 (10), 215 (18), 174 (8), 162 (21), 124 (13), 94 (6).

Tartaric Acid Salt of 3

The above compound 3 (300 mg, 0.697 mmol) was suspended in water (RO type) (15 mL) and tartaric acid (105 mg, 0.697 mmol) added. The mixture was stirred for 30 mins before addition of dimethylsulfoxide (AR grade) (15 mL). The resulting solution was stored at −20° C.

The structures for compounds 1, 2 and 3 are as follows:

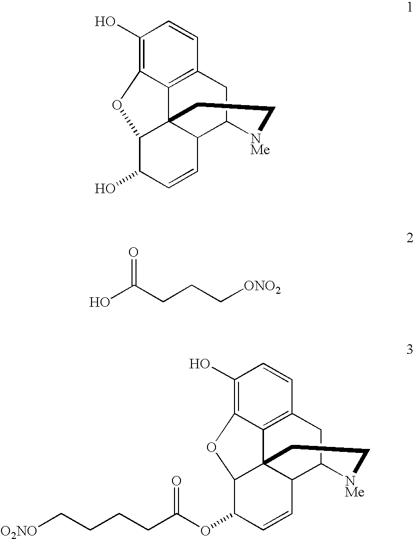

### Example 4

Preparation of Morphine-Nitric Oxide Conjugate 2

5-Nitratovaleroyl Chloride 4

The titled compound was prepared following the procedure of EP 0 984 012 A2 (K. M. Lundy, M. T. Clark). Briefly, 5-nitratovaleric acid (13.34 g, 0.082 mol) was pre-dried under high vacuum (0.01 mmHg) and subsequently dissolved in anhydrous dichloromethane (200 mL) under an argon atmosphere. To this was added phosphorous pentachloride (17.03 g, 0.082 mol) portionwise over 2 mins. The mixture was stirred for 15 h at room temperature. The solvent and excess hydrochloric acid was removed in vacuo and the residue dissolved in anhydrous toluene. The toluene was then 90% removed by distillation under argon at atmospheric pressure. [Warning: distillation must not be allowed to completely remove toluene as this will result in spontaneous explosive decomposition] Toluene is essential for removal of phosphorous oxy chloride. The toluene acid chloride mixture was used without further purification.

Morphine NO Donor 5

Morphine hydrochloride trihydrate (50 mg, 0.133 mmol) and 5-nitratovaleroyl chloride 4 (169 mg, 0.931 mmol) were heated together neat at 135° C. for 7 mins which affords a homogeneous mixture. On cooling the liquid is diluted with dichloromethane (10 mL) and transferred to a separatory funnel containing saturated sodium hydrogen carbonate solution (20 mL). After several washings the organic layer was dried (Na₂SO₄) and evaporated. The residue was subjected to column chromatography (ethyl acetate/methanol, gradient) on silica affording the morphine NO Donor 5 as a brown oil. ¹H n.m.r (200 MHz) 1.60-2.01 (m, 12H), 2.25-2.71 (m, 4H), 2.65 (s, 3H), 2.89-3.28 (m, 3H), 3.65-3.75 (m, 1H), 4.35-4.55 (m, 4H), 5.09-5.25 (m, 2H), 5.32-5.45 (m, 1H), 5.60-5.71 (m, 1H), 6.55-6.85 (m, 2H). Mass spectrum m/z (EI) 575 (M^{+•}, 6%), 548 (1), 530 (1), 503 (1), 472 (1), 454 (1), 430 (1), 403 (1), 385 (1), 354 (1), 285 (20), 268 (60), 215 (22), 162 (20), 146 (13), 124 (13), 100 (24), 81 (19), 42(100).

The structures for the compounds 4 and 5 are as follows:

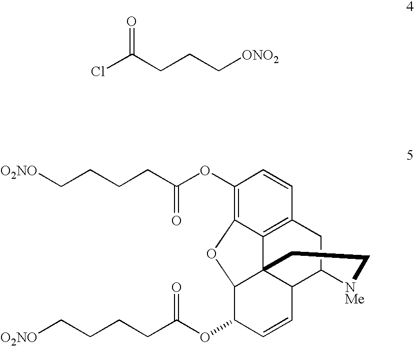

### Example 5

Preparation of Oxcodone-Nitric Oxide Conjugate

Oxycodone 6

Oxycodone hydrochloride (1.5 g) was dissolved in the minimum amount of water (RO type) (˜20 mL) and to this was added enough saturated sodium hydrogen carbonate to raise the pH of the solution to about 11 and to precipitate oxycodone. Oxycodone 6 was collected by vacuum filtration and washed with distilled water (20 mL) followed by small amounts of cold diethyl ether (5 mL). The white solid, protected from light with aluminium foil, was placed under high vacuum (0.01 mm Hg) for 3 h.

Oxycodone NO Donor 7

Freshly prepared oxycodone 6 (500 mg, 1.59 mmol), dicyclohexylcarbodiimide (327 mg, 1.59 mmol), and 5-nitratovaleric acid 2 (259 mg, 1.59 mmol) were dissolved in anhydrous chloroform (90 mL) under an argon atmosphere. The mixture was refluxed for 12 h and allowed to cool. Additional dicyclohexylcarbodiimide (327 mg, 1.59 mmol), and 5-nitratovaleric acid (259 mg, 1.59 mmol) were added and refluxing continued for 6 h. On cooling the solvent was removed in vacuo and the residue dissolved in a solution of warmed ethyl acetate (˜5 mL) and filtered to remove N,N-dicyclohexylurea. The filtrate was concentrated and subjected to column chromatography (ethyl acetate/dichloromethane; gradient) on silica gel which affords derivative 7 as a pale yellow solid.

Tartaric Acid Salt of 7

The above compound 7 (300 mg, 0.651 mmol) was suspended in water (RO type) (15 mL) and tartaric acid (98 mg, 0.651 mmol) added. The mixture was stirred for 30 mins before addition of dimethylsulfoxide (AR grade) (15 mL). The resulting solution was stored at −20° C.

The structures for compounds 6 and 7 are as follows:

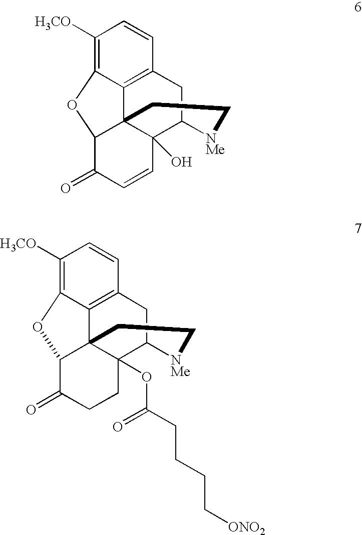

The disclosure of every patent, patent application, and publication cited herein is hereby incorporated herein by reference in its entirety.

The citation of any reference herein should not be construed as an admission that such reference is available as “Prior Art” to the instant application

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

TABLE 1
STZ-induced diabetes in DA rats produces a rightward shift
in the dose-response curve for morphine and oxycodone by
3 wks post-STZ administration

Mean ( ±SEM) ED₅₀

| | Oxycodone (mg/kg) | Morphine (mg/kg) |
|---|---|---|
| Control naïve non-diabetic | 1.2 ± 0.1 | 2.4 ± 0.3 |
| rats | | |
| STZ-diabetic rats | 2.0 ± 0.15* | 6.1 ± 0.3* |
| 3 Wks | | |
| STZ-diabetic rats | 2.1 ± 0.4* | 6.1 ± 0.4* |
| 9 Wks | | |
| STZ-diabetic rats | 4.1 ± 0.3* | No efficacy |
| 12 Wks | | |
| STZ-diabetic rats | 4.2 ± 0.3* | No efficacy |
| 24 Wks | | |

What is claimed is:

1. A method for producing analgesia in a subject having, or at risk of developing, reduced analgesic sensitivity to an opioid receptor agonist, the method comprising administering separately, simultaneously or sequentially to the subject a nitric oxide donor in an amount that is effective for preventing, attenuating and/or reversing the reduced analgesic sensitivity, and an opioid analgesic, which agonises the same opioid receptor as the opioid receptor agonist that is the subject of the reduced analgesic sensitivity, in an amount that is effective for producing the analgesia.

2. A method according to claim 1, wherein the nitric oxide donor is selected from the group consisting of a compound that is converted into nitric oxide, a compound that is degraded or metabolised into nitric oxide and a compound that provides a source of in vivo nitric oxide.

3. A method according to claim 1, wherein the nitric oxide donor is selected from the group consisting of L-arginine, sodium nitroprusside, nitroglycerine, glyceryl trinitrate, isosorbide mononitrate, isosorbide dinitrate, S-nitroso-N-acetyl-penicillamine, a pseudojujubogenin glycoside, a dammarane-type triterpenoid saponin, and an analogue, derivative and pharmaceutically compatible salt of any one of these.

4. A method according to claim 1, wherein the nitric oxide donor is L-arginine or an analogue or derivative thereof.

5. A method according to claim 1, wherein the opioid analgesic is selected from the group consisting of a μ-opioid receptor agonist, a compound which is metabolised to a μ-opioid receptor agonist and a compound that is converted in vivo to a μ-opioid receptor agonist.

6. A method according to claim 5, wherein the μ-opioid receptor agonist is selected from the group consisting of morphine, methadone, fentanyl, sufentanil, alfentanil, hydromorphone, oxymorphone, and an analogue, derivative, prodrug and pharmaceutically compatible salt of any one of these.

7. A method according to claim 5, wherein the μ-opioid receptor agonist is selected from morphine, a morphine analogue, a morphine derivative, a morphine prodrug, and a pharmaceutically compatible salt of any one of these.

8. A method according to claim 1, wherein the opioid receptor agonist is selected from the group consisting of a κ₂-opioid receptor agonist, a compound which is metabolised to a κ₂-opioid receptor agonist and a compound that is converted in vivo to a κ₂-opioid receptor agonist.

9. A method according to claim 8, wherein the κ₂-opioid receptor agonist is metabolised or otherwise converted in vivo to a μ-opioid receptor agonist.

10. A method according to claim 8, wherein the κ₂-opioid receptor agonist is selected from oxycodone, an oxycodone analogue, an oxycodone derivative, an oxycodone prodrug, and a pharmaceutically compatible salt of any one of these.

11. A method according to claim 1, wherein the nitric oxide donor and the opioid analgesic are administered in the form of a single composition.

12. A method according to claim 11, wherein the nitric oxide donor and the opioid analgesic are in the form of separate compounds.

13. A method according to claim 11, wherein the nitric oxide donor and the opioid analgesic are in the form of a conjugate.

14. A method according to claim 1, wherein the reduced analgesic sensitivity is associated with a neuropathic condition.

15. A method according to claim 14, wherein the neuropathic condition is a primary neuropathic condition.

16. A method according to claim 14, wherein the neuropathic condition is a peripheral neuropathic condition.

17. A method according to claim 14, wherein the neuropathic condition is a painful diabetic neuropathy (PDN).

18. A method according to claim 1, wherein the nitric oxide donor and the opioid analgesic are each administered by a route selected from the group consisting of: injecting parenterally including intramuscular, subcutaneous, intramedullary, intrathecal, intraventricular, intravenous, intraperitoneal, and intraocular routes; applying topically including epithelial, and mucosal delivery such as rectal, vaginal, and intranasal routes; and delivering orally.

19. A method according to claim 1, wherein the nitric oxide donor and the opioid analgesic are each administered orally.

20. A method according to claim 1, wherein the nitric oxide donor and the opioid analgesic are each formulated for sustained release in the subject.

21. A method according to claim 1, wherein the nitric oxide donor and the opioid analgesic are each administered together with a pharmaceutically acceptable carrier and/or diluent.

22. A method for producing analgesia in a subject having or at risk of developing a neuropathic condition, the method comprising administering to the subject a nitric oxide donor in an amount that is effective for preventing, attenuating or reversing a reduced analgesic sensitivity, and an opioid analgesic.

23. A method according to claim 22, wherein the opioid analgesic is an agent to which the subject has reduced analgesic sensitivity.

24. A method according to claim 22, wherein the opioid analgesic is administered in an amount that is effective for the production of analgesia.

25. A method according to claim 22, wherein the neuropathic condition is associated with the development of reduced analgesic sensitivity to an opioid receptor agonist.

26. A method according to claim 25, wherein the opioid analgesic agonises the same opioid receptor as the opioid receptor agonist.

27. A method according to claim 22, wherein the neuropathic condition is a primary neuropathic condition.

28. A method according to claim 22, wherein the neuropathic condition is a peripheral neuropathic condition.

29. A method according to claim 22, wherein the neuropathic condition is a painful diabetic neuropathy (PDN).

30. A method according to claim 29, wherein the neuropathic condition is associated with a disorder selected from the group consisting of diabetes, uraemia, amyloidosis, tumaculous neuropathy, nutritional deficiency and kidney failure.

31. A method according to claim 22, wherein the neuropathic condition is selected from the group consisting of hereditary motor and sensory neuropathies (HMSN), hereditary sensory neuropathies (HSNs), hereditary sensory and autonomic neuropathies, and hereditary neuropathies with ulcero-mutilation.

32. A method according to claim 22, wherein the neuropathic condition is associated with a repetitive activity selected from the group consisting of typing and working on an assembly line.

33. A method according to claim 22, wherein the neuropathic condition is associated with trauma.

34. A method according to claim 22, wherein the neuropathic condition is associated with administering to the subject a medication selected from the group consisting of an AIDS medication, an antibiotic, a gold compound, and a chemotherapeutic agent.

35. A method according to claim 34, wherein the medication is selected from the group consisting of nitrofurantoin, dideoxycytosine, dideoxyinosine, metronidazole, vincristine, and cis-platin.

36. A method according to claim 22, wherein the neuropathic condition is associated with exposing the subject to a chemical compound selected from the group consisting of an alcohol, a lead compound, an arsenic compound, a mercury compound, and an organophosphate compound.

37. A method according to claim 22, wherein the condition is associated with an infectious process.

38. A method according to claim 37, wherein the infectious process is selected from the group consisting of Guillian-Barre syndrome HIV and Herpes Zoster (shingles).

39. A method for preventing, attenuating or reversing the development of analgesic hyposensitivity to an opioid receptor agonist in a subject, the method comprising administering to the subject a nitric oxide donor in an amount that is effective for the prevention, attenuation or reversal of the analgesic hyposensitivity to the opioid receptor agonist.

40. A method for producing analgesia in a subject having, or at risk of developing, reduced analgesic sensitivity to an opioid receptor agonist, the method comprising administering to the subject a nitric oxide donor and an opioid analgesic.

41. A method according to claim 40, wherein the opioid analgesic is the opioid receptor agonist.

42. A method according to claim 40, wherein the nitric oxide donor is administered in an amount that is effective for reversing the development of analgesic hyposensitivity to the opioid receptor agonist.

43. A method according to claim 40, wherein the nitric oxide donor is administered in an amount that is effective for reversing the development of tolerance to the opioid receptor agonist.

44. A method according to claim 40, wherein the subject is afflicted with or at risk of developing a neuropathic condition.

45. A method according to claim 40, wherein the neuropathic condition is a peripheral neuropathic condition.

46. A method according to claim 45, wherein the neuropathic condition is PDN.

47. A method according to claim 40, further comprising administering a pharmaceutically acceptable carrier and/or diluent.

48. A method according to claim 40, wherein the opioid analgesic is selected from the group consisting of a μ-opioid receptor agonist, a compound which is metabolised to a μ-opioid receptor agonist and a compound that is converted in vivo to a μ-opioid receptor agonist.

49. A method according to claim 48, wherein the μ-opioid receptor agonist is selected from morphine, methadone, fentanyl, sufentanil, alfentanil, hydromorphone, oxymorphone, and an analogue, derivative, prodrug and a pharmaceutically compatible salt of any one of these.

50. A method according to claim 48, wherein the μ-opioid receptor agonist is selected from morphine, a morphine analogue, a morphine derivative, a morphine prodrug, and a pharmaceutically compatible salt of any one of these.

51. A method according to claim 40, wherein the opioid analgesic is selected from the group consisting of a κ₂-opioid receptor agonist, a compound which is metabolised to a κ₂-opioid receptor agonist and a compound that is converted in vivo to a κ₂-opioid receptor agonist.

52. A method according to claim 40, wherein the κ₂-opioid receptor agonist is metabolised or otherwise converted in vivo to a μ-opioid receptor agonist.

53. A method according to claim 52, wherein the κ₂-opioid receptor agonist is selected from oxycodone, an oxycodone analogue, an oxycodone derivative, an oxycodone prodrug, and a pharmaceutically compatible salt of any one of these.

54. A method according to claim 40, wherein the opioid analgesic is morphine.

55. A method according to claim 40, wherein the opioid analgesic is an oxycodone.

56. A method according to claim 40, wherein the nitric oxide donor and the opioid analgesic are administered separately.

57. A method according to claim 40, wherein the nitric oxide donor and the opioid analgesic are administered in a composition in combination.

58. A method according to claim 57, wherein the nitric oxide donor and the opioid analgesic are administered simultaneously.

59. A method according to claim 40, wherein the subject suffers from reduced opioid analgesic sensitivity.

60. A method according to claim 40, wherein the subject suffers from the development of tolerance to the opioid receptor agonist.

61. A method of preventing or reversing the development of analgesic hyposensitivity to an opioid receptor agonist in a subject, the method comprising administering a nitric oxide donor and the opioid receptor agonist.

62. A method of preventing or reversing the development of tolerance to an opioid receptor agonist in a subject, the method comprising administering a nitric oxide donor and the opioid receptor agonist.

63. A method according to claim 61 or claim 62, wherein the nitric oxide donor and the opioid receptor agonist are administered in combination in a composition which further comprises a pharmaceutically acceptable carrier.

64. An analgesic composition comprising a nitric oxide donor and an opioid analgesic, each in an amount effective to produce analgesia in a subject having or at risk of developing reduced analgesic sensitivity to an opioid receptor agonist.

65. A composition according to claim 64, wherein the nitric oxide donor is selected from the group consisting of a compound that is converted into nitric oxide, a compound that is degraded or metabolised into nitric oxide and a compound that provides a source of in vivo nitric oxide.

66. A composition according to claim 64, wherein the nitric oxide donor is selected from the group consisting of L-arginine, sodium nitroprusside, nitroglycerine, glyceryl trinitrate, isosorbide mononitrate, isosorbide dinitrate, S-nitroso-N-acetyl-penicillamine, pseudojujubogenin glycosides, dammarane-type triterpenoid saponins, their analogues or derivatives and a pharmaceutically compatible salt of any one of these.

67. A composition according to claim 66, wherein the nitric oxide donor is L-arginine or an analogue or derivative thereof.

68. A composition according to claim 66, wherein the opioid analgesic agonises the same receptor as the opioid receptor agonist.

69. A composition according to claim 68, wherein the opioid analgesic is the opioid receptor agonist.

70. A composition according to claim 64, wherein the opioid analgesic is selected from the group consisting of a μ-opioid receptor agonist, a compound which is metabolised to a μ-opioid receptor agonist and a compound that is converted in vivo to a μ-opioid receptor agonist.

71. A composition according to claim 70, wherein the μ-opioid receptor agonist is selected from the group consisting of morphine, methadone, fentanyl, sufentanil, alfentanil, hydromorphone, oxymorphone, their analogues, derivatives or prodrugs and a pharmaceutically compatible salt of any one of these.

72. A composition according to claim 70, wherein the μ-opioid receptor agonist is selected from morphine, a morphine analogue, a morphine derivative, a morphine prodrug, and a pharmaceutically compatible salt of any one of these.

73. A composition according to claim 64, wherein the opioid receptor agonist is selected from the group consisting of a κ₂-opioid receptor agonist, a compound which is metabolised to a κ₂-opioid receptor agonist and a compound that is converted in vivo to a κ₂-opioid receptor agonist.

74. A composition according to claim 73, wherein the κ₂-opioid receptor agonist is metabolised or otherwise converted in vivo to a μ-opioid receptor agonist.

75. A composition according to claim 73, wherein the κ₂-opioid receptor agonist is selected from oxycodone, an oxycodone analogue, an oxycodone derivative, an oxycodone prodrug, and a pharmaceutically compatible salt of any one of these.

76. A composition according to claim 64, wherein the nitric oxide donor and the opioid analgesic are in the form of separate compounds.

77. A composition according to claim 64, wherein the nitric oxide donor and the opioid analgesic are in the form of a conjugate.

78. A composition according to claim 64, wherein the nitric oxide donor and the opioid analgesic are in the form of a conjugate selected from the following compounds: 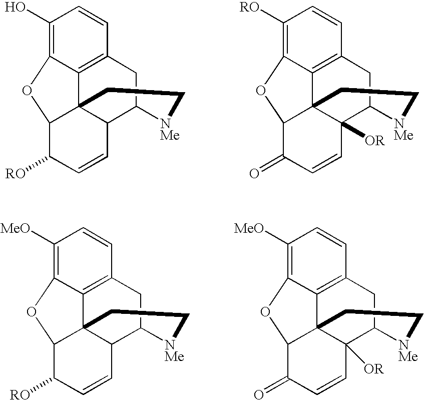
wherein R is H or a group represented by the formula: 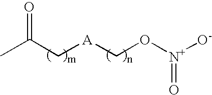
where A is absent or represents a group —O—, —S—, —NH—, —C₆H₄—, —OC₆H₄—, —SC₆H₄— or —NHC₆H₄—;
m is 0 or an integer from 1 to 10; and
n is an integer from 1 to 10 or when A is absent and m is 0, n is an integer from 3 to 10,
and their pharmaceutically compatible salts.

79. A composition according to claim 78, wherein R is a group represented by a formula selected from the group consisting of:

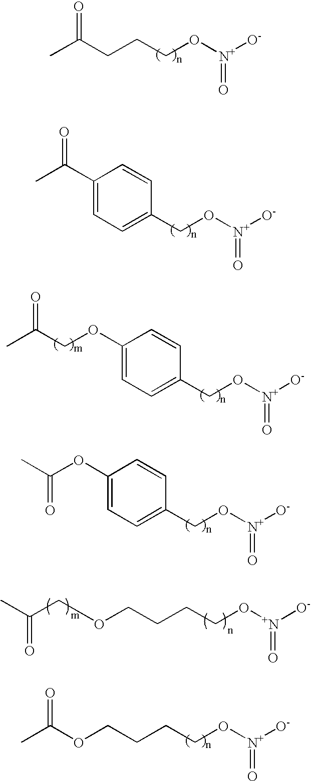

80. A composition according to claim 78, wherein the conjugate is a compound represented by a formula selected from the group consisting of: 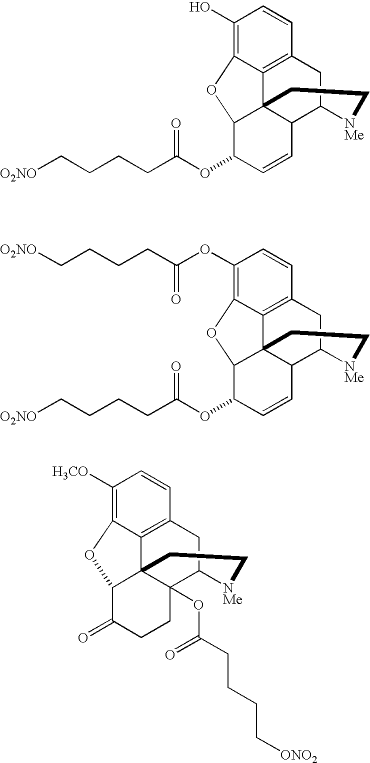
and their pharmaceutically compatible salts.

81. A composition according to claim 64, further comprising a pharmaceutically acceptable carrier.

82. A composition comprising L-arginine and morphine.

83. A composition comprising L-arginine and oxycodone.
